(19) United States
(12) Patent Application Publication
(10) Pub. No.: US 2025/0295745 A1
(43) Pub. Date: Sep. 25, 2025

(54) CROSS-LINKED TUMOR LYSATE SPHERICAL NUCLEIC ACIDS AS CANCER VACCINES

(71) Applicant: NORTHWESTERN UNIVERSITY, Evanston, IL (US)

(72) Inventors: Chad A. Mirkin, Wilmette, IL (US); Michelle Hope Teplensky, Evanston, IL (US); Michael Evangelopoulos, Evanston, IL (US); Alexander Carlos Solivan, Evanston, IL (US)

(21) Appl. No.: 18/864,318

(22) PCT Filed: May 11, 2023
(86) PCT No.: PCT/US23/66881

§ 371 Date: Nov. 8, 2024

Related U.S. Application Data
(60) Provisional application No. 63/340,757, filed on May 11, 2022.

(51) Int. Cl. A61K 39/00; A61K 39/385

(52) U.S. Cl. CPC A61K 39/0011; A61K 39/385; A61K 2039/54; A61K 2039/543; A61K 2039/6025

(57) ABSTRACT

The disclosure is generally related to cross-linked tumor lysate spherical nucleic acids (CLSNAs), nanostructures comprising a core to which a shell of oligonucleotides is attached. Methods of making and using the CLSNAs are also provided herein. In some aspects, the disclosure provides a cross-linked tumor lysate spherical nucleic acid (CLSNA) comprising: (a) a core comprising a plurality of cross-linked tumor cell antigens; and (b) a shell of oligonucleotides attached to the external surface of the core, the shell of oligonucleotides comprising one or more immunostimulatory oligonucleotides.

### CROSS-REFERENCE TO RELATED APPLICATIONS

This application claims the priority benefit under 35 U.S.C. § 119(e) of U.S. Provisional Application No. 63/340,757, filed May 11, 2022, which is incorporated herein by reference in its entirety.

### STATEMENT OF GOVERNMENT INTEREST

This invention was made with government support under grant number 5U54CA199091-05 awarded by the National Institutes of Health. The government has certain rights in the invention.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “2022-036_Seqlisting.XML”, which was created on May 10, 2023 and is 6,434 bytes in size. The subject matter of the Sequence Listing is incorporated herein in its entirety by reference.

### FIELD

The disclosure is generally related to cross-linked tumor lysate spherical nucleic acid (CLSNA). CLSNAs comprise (a) a core comprising a plurality of cross-linked tumor cell antigens; and (b) a shell of oligonucleotides attached to the external surface of the core, the shell of oligonucleotides comprising one or more immunostimulatory oligonucleotides. Methods of making and using the CLSNAs are also provided herein.

### BACKGROUND

Immunotherapy is a promising form of cancer treatment that involves utilizing a patient's own immune system to destroy dangerous and malignant cells in the body with specificity and efficacy (Pardoll, D. M. (2012). The blockade of immune checkpoints in cancer immunotherapy. Nature Reviews. Cancer, 12(4), 252-264). Some immunotherapies, such as checkpoint inhibitor therapy, have shown promise against cancers, including lung cancer (Topalian, S. L.; Drake, C. G.; Pardoll, D. M. Immune Checkpoint Blockade: A Common Denominator Approach to Cancer Therapy. Cancer Cell 2015, 27 (4): 450-461). However, for many other types of cancer, such as pancreatic cancer, checkpoint inhibitor therapy has yet to find the same level of clinical efficacy (Bian, J.; Almhanna, K. Pancreatic cancer and immune checkpoint inhibitors—still a long way to go. Trans Gastroenterol Hepatol 2021, 6:6). Developing treatments to overcome these setbacks has, for example, led to the development of cancer vaccines, where antigen presenting cells (APCs) within the immune system are exposed to peptides or whole proteins from tumors (termed antigen) and stimulatory substances that enhance the antigen-specific immune response (termed adjuvant). The primed APCs then activate the adaptive immune system by stimulating T cells, which have the capacity to kill tumor cells (Fan, Y.; Moon, J. J. Nanoparticle Drug Delivery Systems Designed to Improve Cancer Vaccines and Immunotherapy. Vaccines 2015, 3 (3): 662-685).

### SUMMARY

Cancer vaccines are a form of immunotherapy that utilize adjuvants (immune system stimulators) and tumor-specific antigens (immune system targets) to stimulate a potent response that can identify and kill tumor cells. A major focus of vaccine design has been the selection of tumor specific antigens to produce robust downstream immune responses. However, there is often little consideration of the structural orientation of the components in the vaccine itself and identifying peptides for all tumors is difficult. Spherical nucleic acids (SNAs) are a nucleic acid architecture consisting of a dense shell of oligonucleotides radially oriented around a nanoparticle core. SNAs exhibit unique properties, such as rapid and enhanced cellular uptake and increased resistance to nuclease degradation. By incorporating tumor specific antigens and utilizing immunostimulatory oligonucleotides as the oligonucleotide shell, SNAs can co-deliver vaccine components to immune cells to produce a powerful antitumor response compared to a simple mixture containing the same components. Disclosed herein is a nanoparticle core composed entirely of tumor lysate that takes greater advantage of the particle's total volume and is an efficient vehicle for immunogenic cargo delivery. The present disclosure provides a new powerful and versatile platform for efficient delivery of whole tumor lysate into immune cells using covalent cross-linking chemistry and the SNA architecture, which will improve the treatment of cancers with no known antigenic peptides.

Cross-linking tumor lysate proteins enables generation of a nanoparticle core that can be functionalized with oligonucleotides to form a Spherical Nucleic Acid (SNA) architecture. This structure acts as an immunostimulatory cancer vaccine.

Applications of the technology described herein include, but are not limited to:
Vaccine design
Cancer immunotherapy
Nanomedicine
Immunotherapy for unknown targets

Advantages of the technology provided herein include, but are not limited to:
Ability to deliver large doses of tumor lysate to immune cells
Method of development enables efficient use of tumor lysate
Resistant to enzymatic degradation
Ability to codeliver immune activation and tumor-specific components of a vaccine
Platform technology for personalized medicine

Accordingly, in some aspects the disclosure provides a cross-linked tumor lysate spherical nucleic acid (CLSNA) comprising: (a) a core comprising a plurality of cross-linked tumor cell antigens; and (b) a shell of oligonucleotides attached to the external surface of the core, the shell of oligonucleotides comprising one or more immunostimulatory oligonucleotides. In some embodiments, the plurality of cross-linked tumor cell antigens comprises a tumor cell lysate, purified protein tumor antigens, synthesized tumor antigens, or a combination thereof. In further embodiments, the core comprises about 0.25 femtograms (fg)-2 fg of cross-linked tumor cell antigens. In various embodiments, at least one of the one or more immunostimulatory oligonucleotide comprises a CpG nucleotide sequence. In some embodiments, at least one of the one or more immunostimulatory oligonucleotides is a toll-like receptor (TLR) agonist. In some embodiments, each of the one or more immunostimulatory oligonucleotides is a toll-like receptor (TLR) agonist. In various embodiments, the TLR agonist is a toll-like receptor 1 (TLR1) agonist, a toll-like receptor 2 (TLR2) agonist, a toll-like receptor 3 (TLR3) agonist, a toll-like receptor 4 (TLR4) agonist, a toll-like receptor 5 (TLR5) agonist, a toll-like receptor 6 (TLR6) agonist, a toll-like receptor 7 (TLR7) agonist, a toll-like receptor 8 (TLR8) agonist, a toll-like receptor 9 (TLR9) agonist, a toll-like receptor 10 (TLR10) agonist, a toll-like receptor 11 (TLR11) agonist, a toll-like receptor 12 (TLR12) agonist, a toll-like receptor 13 (TLR13) agonist, or a combination thereof. In further embodiments, the TLR agonist is a toll-like receptor 3 (TLR3) agonist, a toll-like receptor 7 (TLR7) agonist, a toll-like receptor 8 (TLR8) agonist, a toll-like receptor 9 (TLR9) agonist, or a combination thereof. In some embodiments, one or more oligonucleotides in the shell of oligonucleotides comprises or consists of the sequence of 5′-TCCATGACGTTCCTGACGTT-3′ (SEQ ID NO: 2). In further embodiments, one or more oligonucleotides in the shell of oligonucleotides comprises or consists of the sequence of 5′-TCGTCGTTTTGTCGTTTTGTCGTT-3′ (SEQ ID NO: 3). In still further embodiments, one or more oligonucleotides in the shell of oligonucleotides comprises or consists of the sequence of 5′-TCCATGACGTTCCTGACGTT (Spacer-18 (hexaethyleneglycol))₂dibenzocyclooctyl (DBCO)-3′ (SEQ ID NO: 4). In some embodiments, one or more oligonucleotides in the shell of oligonucleotides comprises or consists of the sequence of 5′-TCGTCGTTTTGTCGTTTTGTCGTT (Spacer-18 (hexaethyleneglycol))₂dibenzocyclooctyl (DBCO)-3′ (SEQ ID NO: 5). In various embodiments, one or more oligonucleotides in the shell of oligonucleotides is modified on its 5′ end and/or 3′ end with dibenzocyclooctyl (DBCO). In further embodiments, one or more oligonucleotides in the shell of oligonucleotides is modified on its 5′ end and/or 3′ end with a thiol. In some embodiments, one or more oligonucleotides in the shell of oligonucleotides is modified on its 5′ end and/or 3′ end with a maleimide. In still further embodiments, one or more oligonucleotides in the shell of oligonucleotides is modified on its 5′ end and/or 3′ end with an azide. In various embodiments, diameter of the CLSNA is about 20 nanometers (nm) to about 300 nm. In further embodiments, diameter of the CLSNA is less than or equal to about 300 nanometers.

In still further embodiments, diameter of the nanoparticle is less than or equal to about 170 nanometers. In various embodiments, the CLSNA comprises about 10 to about 750 oligonucleotides. In further embodiments, the CLSNA comprises about 200 to about 300 oligonucleotides. In various embodiments, the plurality of cross-linked tumor cell antigens is derived from a tumor lysate exposed to a crosslinking agent. In various embodiments, the crosslinking agent is an amine to amine, amine to thiol, thiol to thiol crosslinking agent, or a combination thereof. In some embodiments, the amine to amine crosslinking agent is DSS (disuccinimidyl suberate), BS3 (bis(sulfosuccinimidyl)suberate), DSBU (Disuccinimidyl Dibutyric Urea), DFDNB (1,5-difluoro-2,4,dinitrobenzene), DMP (dimethyl pimelimidate), DMS (dimethyl suberimidate), DSG (disuccinimidyl glutarate), DSP (dithiobis(succinimidyl propionate)), DSSO (disuccinimidyl sulfoxide), DST (disuccinimidyl tartrate), DTBP (dimethyl 3,3′-dithiobispropionimidate), DTSSP (3,3′-dithiobis(sulfosuccinimidyl propionate)), EGS (ethylene glycol bis(succinimidyl succinate)), Sulfo-EGS (ethylene glycol bis(sulfosuccinimidyl succinate)), TSAT (tris-(succinimidyl)aminotriacetate), or a combination thereof. In some embodiments, the amine to thiol crosslinking agent is Sulfo-SMCC (sulfosuccinimidyl 4-(N-maleimidomethyl)cyclohexane-1-carboxylate), SM(PEG)2 (PEGylated SMCC crosslinker), BMPS (N-β-maleimidopropyl-oxysuccinimide ester), AMAS (N-α-maleimidoacet-oxysuccinimide ester), EMCS (N—ε-malemidocaproyl-oxysuccinimide ester), GMBS (N-γ-maleimidobutyryl-oxysuccinimide ester), LC-SMCC (succinimidyl 4-(N-maleimidomethyl)cyclohexane-1-carboxy-(6-amidocaproate)), LC-SPDP (succinimidyl 6-(3(2-pyridyldithio)propionamido)hexanoate), MBS (m-maleimidobenzoyl-N-hydroxysuccinimide ester), PEG4-SPDP (PEGylated, long-chain SPDP crosslinker), SBAP (succinimidyl 3-(bromoacetamido)propionate), SIA (succinimidyl iodoacetate), SIAB (succinimidyl (4-iodoacetyl)aminobenzoate), SM(PEG)12 (PEGylated, long-chain SMCC crosslinker), SMPB (succinimidyl 4-(β-maleimidophenyl)butyrate), SMPH (Succinimidyl 6-((beta-maleimidopropionamido)hexanoate)), SMPT (4-succinimidyloxycarbonyl-alpha-methyl-α(2-pyridyldithio)toluene), SPDP (succinimidyl 3-(2-pyridyldithio)propionate), Sulfo-EMCS (N-ε-maleimidocaproyl-oxysulfosuccinimide ester), Sulfo-GMBS (N-γ-maleimidobutyryl-oxysulfosuccinimide ester), Sulfo-KMUS (N-κ-maleimidoundecanoyl-oxysulfosuccinimide ester), Sulfo-MBS (m-maleimidobenzoyl-N-hydroxysulfosuccinimide ester), Sulfo-SIAB (sulfosuccinimidyl (4-iodoacetyl)aminobenzoate), Sulfo-SMPB (sulfosuccinimidyl 4-(N-maleimidophenyl)butyrate), or a combination thereof. In some embodiments, the thiol to thiol crosslinking agent is BM(PEG)3 (1,11-bismaleimido-triethyleneglycol), BMB (1,4-bismaleimidobutane), BMH (bismaleimidohexane), BMOE (bismaleimidoethane), DTME (dithiobismaleimidoethane), TMEA (tris(2-maleimidoethyl)amine), or a combination thereof. In various embodiments, the plurality of cross-linked tumor cell antigens is derived from a breast cancer cell, peritoneum cancer cell, cervical cancer cell, colon cancer cell, rectal cancer cell, esophageal cancer cell, eye cancer cell, liver cancer cell, pancreatic cancer cell, larynx cancer cell, lung cancer cell, skin cancer cell, ovarian cancer cell, prostate cancer cell, stomach cancer cell, testicular cancer cell, thyroid cancer cell, brain cancer cell, or a combination thereof. In various embodiments, the shell of oligonucleotides comprises DNA oligonucleotides, RNA oligonucleotides, or a combination thereof. In some embodiments, the shell of oligonucleotides comprises DNA oligonucleotides and RNA oligonucleotides. In further embodiments, the shell of oligonucleotides comprises single-stranded DNA, double-stranded DNA, single-stranded RNA, double-stranded RNA, or a combination thereof. In various embodiments, the shell of oligonucleotides comprises a targeting oligonucleotide, an inhibitory oligonucleotide, a non-targeting oligonucleotide, or a combination thereof. In further embodiments, the inhibitory oligonucleotide is an antisense oligonucleotide, small interfering RNA (siRNA), an aptamer, a short hairpin RNA (shRNA), a DNAzyme, or an aptazyme. In various embodiments, one or more oligonucleotides in the shell of oligonucleotides is a modified oligonucleotide. In various embodiments, each tumor cell antigen in the plurality of cross-linked tumor cell antigens is the same, or wherein at least two tumor cell antigens in the plurality of cross-linked tumor cell antigens are different.

In further aspects, the disclosure provides a pharmaceutical formulation comprising the CLSNA of the disclosure and a pharmaceutically acceptable carrier or diluent.

In some aspects, the disclosure provides a method of making a cross-linked tumor lysate spherical nucleic acid (CLSNA) comprising: contacting one or more tumor antigens with a crosslinking agent to produce a core comprising a plurality of cross-linked tumor cell antigens; then contacting the core with one or more oligonucleotides to make the CLSNA, wherein the core and the one or more oligonucleotides comprise complementary reactive moieties that together form a covalent bond. In some embodiments, the crosslinking agent is formaldehyde or paraformaldehyde. In some embodiments, the reactive moiety on the core comprises an azide, an alkyne, a maleimide, a thiol, an alcohol, an amine, a carboxylic acid, an olefin, an isothiocyanate, a N-hydroxysuccinimide, a phosphine, a nitrone, a norbornene, an oxanorbornene, a transcycloctene, an s-tetrazene, an isocyanide, a tetrazole, a nitrile oxide, a quadricyclane, or a carbodiimide. In further embodiments, the reactive moiety is on a terminus of each of the one or more oligonucleotides. In still further embodiments, the reactive moiety on the one or more oligonucleotides comprises an alkyne, an azide, a maleimide, a thiol, an alcohol, an amine, a carboxylic acid, an olefin, an isothiocyanate, a N-hydroxysuccinimide, a phosphine, a nitrone, a norbornene, an oxanorbornene, a transcycloctene, an s-tetrazene, an isocyanide, a tetrazole, a nitrile oxide, a quadricyclane, or a carbodiimide. In some embodiments, the alkyne comprises dibenzocyclooctyl (DBCO) alkyne or a terminal alkyne. In further embodiments, the core comprises an azide reactive moiety and each of the one or more oligonucleotides comprises an alkyne reactive moiety, or vice versa. In some embodiments, the alkyne reactive moiety comprises a DBCO alkyne. In some embodiments, the one or more oligonucleotides comprises at least one Toll-Like Receptor (TLR) agonist. In further embodiments, the TLR agonist is a toll-like receptor 1 (TLR1) agonist, a toll-like receptor 2 (TLR2) agonist, a toll-like receptor 3 (TLR3) agonist, a toll-like receptor 4 (TLR4) agonist, a toll-like receptor 5 (TLR5) agonist, a toll-like receptor 6 (TLR6) agonist, a toll-like receptor 7 (TLR7) agonist, a toll-like receptor 8 (TLR8) agonist, a toll-like receptor 9 (TLR9) agonist, a toll-like receptor 10 (TLR10) agonist, a toll-like receptor 11 (TLR11) agonist, a toll-like receptor 12 (TLR12) agonist, a toll-like receptor 13 (TLR13) agonist, or a combination thereof. In some embodiments, the TLR agonist is a toll-like receptor 3 (TLR3) agonist, a toll-like receptor 7 (TLR7) agonist, a toll-like receptor 8 (TLR8) agonist, a toll-like receptor 9 (TLR9) agonist, or a combination thereof. In various embodiments, at least one of the one or more oligonucleotides comprises RNA or DNA. In further embodiments, at least one of the one or more oligonucleotides is DNA. In some embodiments, at least one of the one or more oligonucleotides is a modified oligonucleotide. In various embodiments, the ratio of oligonucleotide to cross-linked tumor cell antigens is about 1:1 to about 1:2.

In further aspects, the disclosure provides a vaccine comprising a CLSNA or pharmaceutical formulation as described herein. In some embodiments, the vaccine comprises an adjuvant.

In some aspects, the disclosure provides an antigenic composition comprising a CLSNA of the disclosure in a pharmaceutically acceptable carrier, diluent, stabilizer, preservative, or adjuvant, or a pharmaceutical formulation or vaccine of the disclosure, wherein the antigenic composition is capable of generating an immune response including antibody generation, an antitumor response, and/or a protective immune response in a mammalian subject. In some embodiments, the antibody response is a neutralizing antibody response or a protective antibody response.

In further aspects, the disclosure provides a method of producing an immune response in a subject having cancer or at risk of developing cancer, comprising administering to the subject an effective amount of a CLSNA, pharmaceutical formulation, vaccine, or antigenic composition of the disclosure, thereby producing the immune response in the subject. In various embodiments, the cancer is breast cancer, peritoneum cancer, cervical cancer, colon cancer, rectal cancer, esophageal cancer, eye cancer, liver cancer, pancreatic cancer, larynx cancer, lung cancer, skin cancer, ovarian cancer, prostate cancer, stomach cancer, testicular cancer, thyroid cancer, brain cancer, or a combination thereof.

In some aspects, the disclosure provides a method of treating a cancer in a subject in need thereof, comprising administering to the subject an effective amount of a CLSNA, pharmaceutical formulation, vaccine, or antigenic composition of the disclosure, thereby treating the cancer in the subject. In various embodiments, the cancer is breast cancer, peritoneum cancer, cervical cancer, colon cancer, rectal cancer, esophageal cancer, eye cancer, liver cancer, pancreatic cancer, larynx cancer, lung cancer, skin cancer, ovarian cancer, prostate cancer, stomach cancer, testicular cancer, thyroid cancer, brain cancer, or a combination thereof. In some embodiments, the administering is subcutaneous. In further embodiments, the administering is intravenous, intraperitoneal, intranasal, or intramuscular.

### BRIEF DESCRIPTION OF THE DRAWINGS

FIG. 1 shows a schematic for the synthesis of cross-linked tumor lysate spherical nucleic acids (CLSNA).

FIG. 2 shows cross-Linked Spherical Nucleic Acid (CLSNA) particle characterization.

FIG. 3 shows results of a degradation study.

FIG. 4 shows results of in vitro uptake studies.

FIG. 5 shows results of in vitro activation studies.

### DETAILED DESCRIPTION

A major focus in cancer vaccine design has been the selection of tumor-specific targets that can produce a robust downstream immune response. However, for many cancers, identifying precise targets is difficult. This has led to the use of tumor lysate (a mixture of all proteins in a tumor cell) as a form of targeting for training the immune system. However, delivery of tumor lysate has many challenges, namely that it is difficult to deliver large quantities of lysate efficiently to immune cells. Moreover, delivery of lysate free in solution fails to take into consideration the structural orientation of the vaccine components, which has been shown to dramatically impact the resulting immune response.

Spherical Nucleic Acids (SNAs) are nanostructures capable of acting as cancer immunotherapeutics and consist of a nanoparticle core surrounded by a dense shell of one or more immunostimulatory oligonucleotides. As disclosed herein, SNAs employing tumor lysate as a key component of the core (i.e., CLSNAs) exhibit the advantageous properties of the SNA, including high resistance to degradation and increased uptake into immune cells. The present disclosure provides a powerful and versatile platform for efficient delivery of tumor lysate into immune cells, which improves the treatment of cancers (e.g., cancers with no known specific targets).

Thus, in some embodiments, the present disclosure provides a system that can rapidly produce potent vaccines for cancers without known tumor specific targets. The technology takes full advantage of the vaccine delivery volume, as the entire volume is an active ingredient (e.g., immunostimulatory components, targeting components). Furthermore, biopsies from patients could be easily employed and developed into this platform, taking full advantage of material already being taken from the patient and providing a platform for personalized medicine.

In further embodiments, the present disclosure allows for the scalable production of potent vaccines for cancers without known tumor specific targets. The platform could be used to take advantage of patient specific tumor biopsies, maximizing the value of the material, to produce personalized cancer vaccines in a shortened timeframe without the need for extensive ex vivo cell handling. Furthermore, the chemistry employed in this platform can be utilized with all types of cell lysate.

As used in this specification and the appended claims, the singular forms “a,” “an,” and “the” include plural reference unless the context clearly dictates otherwise.

All language such as “from,” “to,” “up to,” “at least,” “greater than,” “less than,” and the like include the number recited and refer to ranges which can subsequently be broken down into sub-ranges.

A range includes each individual member. Thus, for example, a group having 1-3 members refers to groups having 1, 2, or 3 members. Similarly, a group having 6 members refers to groups having 1, 2, 3, 4, or 6 members, and so forth.

“About” and “approximately” shall generally mean an acceptable degree of error for the quantity measured given the nature or precision of the measurements. Exemplary degrees of error are within 20-25 percent (%), for example, within 20 percent, 10 percent, 5 percent, 4 percent, 3 percent, 2 percent, or 1 percent of the stated value or range of values.

A “subject” is a vertebrate organism. The subject can be a non-human mammal (e.g., a mouse, a rat, or a non-human primate), or the subject can be a human subject.

The terms “administering”, “administer”, “administration”, and the like, as used herein, refer to any mode of transferring, delivering, introducing, or transporting a CLSNA to a subject in need of treatment with such an agent. Such modes include, but are not limited to, oral, topical, intravenous, intraarterial, intraperitoneal, intramuscular, intratumoral, intradermal, intranasal, and subcutaneous administration.

The term “vaccine” as used herein relates to a CLSNA or a composition comprising a CLSNA as described herein that upon administration induces an immune response, for example an antitumor response and/or a cellular immune response, which recognizes and attacks an antigen such as a tumor antigen. A vaccine may be used for the prevention, amelioration, or treatment of a disease (i.e., cancer).

As used herein, “treating” and “treatment” refers to any reduction in the severity and/or onset of symptoms associated with a disease or disorder (i.e., cancer). Accordingly, “treating” and “treatment” includes therapeutic and prophylactic measures. One of ordinary skill in the art will appreciate that any degree of protection from, or amelioration of, the disease (i.e., cancer) is beneficial to a subject, such as a human patient. The quality of life of a patient is improved by reducing to any degree the severity of symptoms in a subject and/or delaying the appearance of symptoms.

As used herein, a “targeting oligonucleotide” is an oligonucleotide that directs a CLSNA to a particular tissue and/or to a particular cell type. In some embodiments, a targeting oligonucleotide is an aptamer. Thus, in some embodiments, a CLSNA of the disclosure comprises an aptamer attached to the exterior of the core, wherein the aptamer is designed to bind one or more receptors on the surface of a certain cell type.

As used herein, an “immunostimulatory oligonucleotide” is an oligonucleotide that can stimulate (e.g., induce or enhance) an immune response. Typical examples of immunostimulatory oligonucleotides are CpG-motif containing oligonucleotides, single-stranded RNA oligonucleotides, double-stranded RNA oligonucleotides, and double-stranded DNA oligonucleotides. A “CpG-motif” is a cytosine-guanine dinucleotide sequence.

In any of the aspects or embodiments of the disclosure, the immunostimulatory oligonucleotide is a toll-like receptor (TLR) agonist (e.g., a toll-like receptor 9 (TLR9) agonist).

The term “inhibitory oligonucleotide” refers to an oligonucleotide that reduces the production or expression of proteins, such as by interfering with translating mRNA into proteins in a ribosome or that are sufficiently complementary to either a gene or an mRNA encoding one or more of targeted proteins, that specifically bind to (hybridize with) the one or more targeted genes or mRNA thereby reducing expression or biological activity of the target protein. Inhibitory oligonucleotides include, without limitation, isolated or synthetic short hairpin RNA (shRNA or DNA), an antisense oligonucleotide (e.g., antisense RNA or DNA, chimeric antisense DNA or RNA), miRNA and miRNA mimics, small interfering RNA (siRNA), DNA or RNA inhibitors of innate immune receptors, an aptamer, a DNAzyme, or an aptazyme.

The term “non-targeting oligonucleotide” refers an oligonucleotide included, in some embodiments, in the shell of oligonucleotides of a CLSNA that is not associated with a particular activity (e.g., an immunostimulatory activity) but instead is used to achieve a certain density of oligonucleotides on the external surface of a CLSNA. Non-limiting examples of non-targeting oligonucleotides are an oligonucleotide comprising a scrambled nucleotide sequence and/or a homopolymeric oligonucleotide (e.g., a polythymidine oligonucleotide (such as T₂₀ (SEQ ID NO: 1))).

An “antigenic composition” is a composition of matter suitable for administration to a human or animal subject (e.g., in an experimental or clinical setting) that is capable of eliciting a specific immune response, e.g., against a tumor cell antigen. In the context of this disclosure, the term antigenic composition will be understood to encompass compositions that are intended for administration to a subject or population of subjects for the purpose of eliciting a protective or palliative immune response against a tumor cell antigen.

The term “dose” as used herein refers to a measured portion of any of the CLSNAs of the disclosure (e.g., a CLSNA, antigenic composition, pharmaceutical formulation as described herein) taken by (administered to or received by) a subject at any one time.

An “immune response” is a response of a cell of the immune system, such as a B cell, T cell, or monocyte, to a stimulus, such as a CLSNA as described herein. An immune response can be an antitumor response. An immune response can also be a B cell response, which results in the production of specific antibodies, such as antigen specific neutralizing antibodies. An immune response can also be a T cell response, such as a CD4⁺ helper T cell response or a CD8⁺ cytotoxic T cell response. B cell and T cell responses are aspects of a “cellular” immune response. As described herein, an “immune response” can also be a “treatment based” response in which the immune system is being primed while actively fighting the tumor. An immune response can also be a “humoral” immune response, which is mediated by antibodies. In some cases, the response is specific for a particular antigen (that is, an “antigen-specific response”). A “protective immune response” is an immune response that inhibits a detrimental function or activity of an antigen, or decreases symptoms (including death) that result from the antigen. Protective in this context does not necessarily require that the subject is completely protected against infection. A protective response is achieved when the subject is protected from developing symptoms of disease, or when the subject experiences a lower severity of symptoms of disease. A protective immune response can be measured, for example, by immune assays using a serum sample from an immunized subject and testing the ability of serum antibodies for inhibition of pseudoviral binding, such as: pseudovirus neutralization assay (or surrogate virus neutralization test), ELISA-neutralization assay, antibody dependent cell-mediated cytotoxicity assay (ADCC), complement-dependent cytotoxicity (CDC), antibody dependent cell-mediated phagocytosis (ADCP), enzyme-linked immunospot (ELISpot). In addition, vaccine efficacy can be tested by measuring B or T cell activation after immunization, using flow cytometry (FACS) analysis or ELISpot assay. The protective immune response can be tested by measuring resistance to antigen challenge in vivo in an animal model. In humans, a protective immune response can be demonstrated in a population study, comparing measurements of symptoms, morbidity, mortality, etc. in treated subjects compared to untreated controls. Exposure of a subject to an immunogenic stimulus, such as a SNA as described herein, elicits a primary immune response specific for the stimulus, that is, the exposure “primes” the immune response. A subsequent exposure, e.g., by immunization, to the stimulus can increase or “boost” the magnitude (or duration, or both) of the specific immune response. Thus, “boosting” a preexisting immune response by administering, e.g., an antigenic composition of the disclosure increases the magnitude of an antigen-specific response, (e.g., by increasing the breadth of produced antibodies (i.e., in the case of administering a booster that primes the immune system against a variant), by increasing antibody titer and/or affinity, by increasing the frequency of antigen specific B or T cells, by inducing maturation effector function, or a combination thereof). The “maturity and memory” of B and T cells may also be measured as an indicator of an immune response.

“Adjuvant” refers to a substance which, when added to a composition comprising a tumor cell antigen, nonspecifically enhances or potentiates an immune response to the antigen in the recipient upon exposure. In any of the aspects or embodiments of the disclosure, the CLSNAs provided herein comprise immunostimulatory oligonucleotides (for example and without limitation, a toll-like receptor (TLR) agonist) as adjuvants and comprise tumor cell antigens as described herein. Additional adjuvants contemplated for use according to the disclosure include aluminum (e.g., aluminum hydroxide), lipid-based adjuvant AS01B, alum, MF59, in addition to TLR agonists as described herein (e.g., CpG DNA, TLR7's imiquimod, TLR8's Motolimod, TLR4's MPLA4, TLR3's Poly (I:C), or a combination thereof).

An “effective amount” or a “sufficient amount” of a substance is that amount necessary to effect beneficial or desired results, including clinical results, and, as such, an “effective amount” depends upon the context in which it is being applied. In the context of administering a CLSNA of the disclosure, for example, an effective amount contains sufficient antigen to elicit an immune response. In some embodiments, an effective amount of CLSNA is an amount sufficient to inhibit gene expression. An effective amount can be administered in one or more doses as described further herein. Efficacy can be shown in an experimental or clinical trial, for example, by comparing results achieved with a substance of interest compared to an experimental control.

All references, patents, and patent applications disclosed herein are incorporated by reference with respect to the subject matter for which each is cited, which in some cases may encompass the entirety of the document.

### CROSS-LINKED TUMOR LYSATE SPHERICAL NUCLEIC ACIDS (CLSNAs)

Spherical nucleic acids (SNAs) generally comprise a nanoparticle core and a shell of oligonucleotides attached to the external surface of the nanoparticle core. The present disclosure provides cross-linked tumor lysate spherical nucleic acids (CLSNAs), which are SNAs comprising a core comprising a plurality of cross-linked tumor cell antigens and a shell of oligonucleotides attached to the external surface of the core. Thus, in a CLSNA the plurality of cross-linked tumor cell antigens serves as the core. Accordingly, in any of the aspects or embodiments of the disclosure, a cross-linked tumor lysate spherical nucleic acid (CLSNA) is provided comprising: (a) a core comprising a plurality of cross-linked tumor cell antigens; and (b) a shell of oligonucleotides attached to the external surface of the core, the shell of oligonucleotides comprising one or more immunostimulatory oligonucleotides.

CLSNAs can range in size from about 1 nanometer (nm) to about 500 nm, about 1 nm to about 400 nm, about 1 nm to about 300 nm, about 1 nm to about 200 nm, about 1 nm to about 150 nm, about 1 nm to about 100 nm, about 1 nm to about 90 nm, about 1 nm to about 80 nm in diameter, about 1 nm to about 70 nm in diameter, about 1 nm to about 60 nm in diameter, about 1 nm to about 50 nm in diameter, about 1 nm to about 40 nm in diameter, about 1 nm to about 30 nm in diameter, about 1 nm to about 20 nm in diameter, about 1 nm to about 10 nm, about 10 nm to about 150 nm in diameter, about 10 nm to about 140 nm in diameter, about 10 nm to about 130 nm in diameter, about 10 nm to about 120 nm in diameter, about 10 nm to about 110 nm in diameter, about 10 nm to about 100 nm in diameter, about 10 nm to about 90 nm in diameter, about 10 nm to about 80 nm in diameter, about 10 nm to about 70 nm in diameter, about 10 nm to about 60 nm in diameter, about 10 nm to about 50 nm in diameter, about 10 nm to about 40 nm in diameter, about 10 nm to about 30 nm in diameter, or about 10 nm to about 20 nm in diameter. In further aspects, the disclosure provides a plurality of CLSNAs, each CLSNA comprising one or more oligonucleotides attached thereto. Thus, in some embodiments, the size of the plurality of CLSNAs is from about 10 nm to about 150 nm (mean diameter), about 10 nm to about 140 nm in mean diameter, about 10 nm to about 130 nm in mean diameter, about 10 nm to about 120 nm in mean diameter, about 10 nm to about 110 nm in mean diameter, about 10 nm to about 100 nm in mean diameter, about 10 nm to about 90 nm in mean diameter, about 10 nm to about 80 nm in mean diameter, about 10 nm to about 70 nm in mean diameter, about 10 nm to about 60 nm in mean diameter, about 10 nm to about 50 nm in mean diameter, about 10 nm to about 40 nm in mean diameter, about 10 nm to about 30 nm in mean diameter, or about 10 nm to about 20 nm in mean diameter. In some embodiments, the diameter (or mean diameter for a plurality of CLSNAs) of the CLSNAs is from about 10 nm to about 150 nm, from about 30 to about 100 nm, or from about 40 to about 80 nm. In some embodiments, the size of the nanoparticles used in a method varies as required by their particular use or application. The variation of size is advantageously used to optimize certain physical characteristics of the CLSNAs, for example, the amount of surface area to which oligonucleotides may be attached as described herein. It will be understood that the foregoing diameters of CLSNAs can apply to the diameter of the core itself or to the diameter of the core and the shell of oligonucleotides attached thereto.

### Tumor Cell Antigens

Cross-linked tumor lysate spherical nucleic acids (CLSNAs) comprise, in various aspects, (a) a core comprising a plurality of cross-linked tumor cell antigens; and (b) a shell of oligonucleotides attached to the external surface of the core, the shell of oligonucleotides comprising one or more immunostimulatory oligonucleotides. Tumor lysates comprise a mixture of all proteins in a tumor cell, and tumor cell antigens, in turn, are derived from (e.g., isolated from) tumor lysates. In any of the aspects or embodiments of the disclosure, and as described herein below, the tumor lysate comprising tumor cell antigens is then cross-linked to form the core of the CLSNA. In any of the aspects or embodiments of the disclosure, the soluble portion of the tumor lysate comprising tumor cell antigens is cross-linked to form the core of the CLSNA. In some embodiments, tumor cell antigens are isolated from a tumor cell lysate and then the isolated tumor cell antigens are cross-linked to form the core of the CLSNA.

Tumor lysates from any tumor cell are contemplated for use according to the disclosure. For example and without limitation, tumor lysates may be obtained from a tumor cell associated with breast cancer, peritoneum cancer, cervical cancer, colon cancer, rectal cancer, esophageal cancer, eye cancer, liver cancer, pancreatic cancer, larynx cancer, lung cancer, skin cancer, ovarian cancer, prostate cancer, stomach cancer, testicular cancer, thyroid cancer, brain cancer, lymphoma, or a combination thereof.

### Methods of Making Cross-Linked Tumor Lysate Spherical Nucleic Acids (CLSNAS)

The disclosure also provides methods of making CLSNAs. Accordingly, in some aspects the disclosure provides a method of making a cross-linked tumor lysate spherical nucleic acid (CLSNA) comprising contacting one or more tumor antigens with a crosslinking agent to produce a core comprising a plurality of cross-linked tumor cell antigens; then contacting the core with one or more oligonucleotides to make the CLSNA, wherein the core and the one or more oligonucleotides comprise complementary reactive moieties that together form a covalent bond. In various embodiments, the core comprises about 0.25 femtograms (fg)-2.0 fg of cross-linked tumor cell antigens. In various embodiments, the core comprises about, at least about, or less than about 0.25, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 fg of cross-linked tumor cell antigens. In some embodiments, a tumor lysate is obtained through lysis of tumor cells. The tumor lysate is then reacted with a crosslinking agent that can cross-link to amino acids in the lysate that contain, e.g., a primary amine. In various embodiments, the molar ratio of crosslinking agent to tumor lysate used for the crosslinking is about 5:1, 10:1, 20:1, 50:1, 75:1, 100:1, 150:1, or 200:1.

As described herein, CLSNAs may be synthesized by conjugating (i) oligonucleotides and (ii) the core comprising a plurality of cross-linked tumor antigens, wherein the oligonucleotides and the core comprise complementary reactive moieties that together form a covalent bond. In some embodiments, DBCO-modified oligonucleotide strands are then covalently conjugated to, e.g., azide groups through Cu-free click chemistry. While DBCO-modified DNA was used in examples herein, other alkyne moieties can be used instead, including a terminal alkyne (HC≡C—) or an internal alkyne (RC≡C—, where R comprises an alkyl). The alkyne moiety can also be attached to the oligonucleotide via a linker. In some embodiments, the reactive moiety on the cross-linked tumor antigen core comprises an azide, an alkyne, a maleimide, a thiol, an alcohol, an amine, a carboxylic acid, an olefin, an isothiocyanate, a N-hydroxysuccinimide, a phosphine, a nitrone, a norbornene, an oxanorbornene, a transcycloctene, an s-tetrazene, an isocyanide, a tetrazole, a nitrile oxide, a quadricyclane, or a carbodiimide. In some embodiments, a cross linker is used to facilitate covalent conjugation of an oligonucleotide to the cross-linked tumor antigen core. For example and without limitation, an azide-PEG-NHS ester cross linker may be used to conjugate to a DBCO on the oligonucleotide and a lysine on the protein (i.e., cross-linked tumor cell antigen) core. In various embodiments, the reactive moiety on the oligonucleotide is on a terminus of the oligonucleotide. In still further embodiments, the reactive moiety on the oligonucleotide comprises an alkyne, an azide, a maleimide, a thiol, an alcohol, an amine, a carboxylic acid, an olefin, an isothiocyanate, a N-hydroxysuccinimide, a phosphine, a nitrone, a norbornene, an oxanorbornene, a transcycloctene, an s-tetrazene, an isocyanide, a tetrazole, a nitrile oxide, a quadricyclane, or a carbodiimide. In some embodiments, the alkyne comprises dibenzocyclooctyl (DBCO) alkyne or a terminal alkyne. In further embodiments, the cross-linked tumor cell antigen core comprises an azide reactive moiety and the oligonucleotide comprises an alkyne reactive moiety, or vice versa. In still further embodiments, the alkyne reactive moiety comprises a DBCO alkyne.

In various embodiments, the crosslinking agent is an amine to amine, amine to thiol, thiol to thiol crosslinking agent, or a combination thereof. In some embodiments, the amine to amine crosslinking agent is DSS (disuccinimidyl suberate), BS3 (bis(sulfosuccinimidyl)suberate), DSBU (Disuccinimidyl Dibutyric Urea), DFDNB (1,5-difluoro-2,4,dinitrobenzene), DMP (dimethyl pimelimidate), DMS (dimethyl suberimidate), DSG (disuccinimidyl glutarate), DSP (dithiobis(succinimidyl propionate)), DSSO (disuccinimidyl sulfoxide), DST (disuccinimidyl tartrate), DTBP (dimethyl 3,3′-dithiobispropionimidate), DTSSP (3,3′-dithiobis(sulfosuccinimidyl propionate)), EGS (ethylene glycol bis(succinimidyl succinate)), Sulfo-EGS (ethylene glycol bis(sulfosuccinimidyl succinate)), TSAT (tris-(succinimidyl)aminotriacetate), or a combination thereof. In further embodiments, the amine to thiol crosslinking agent is Sulfo-SMCC (sulfosuccinimidyl 4-(N-maleimidomethyl)cyclohexane-1-carboxylate), SM(PEG)2 (PEGylated SMCC crosslinker), BMPS (N-β-maleimidopropyl-oxysuccinimide ester), AMAS (N-α-maleimidoacet-oxysuccinimide ester), EMCS (N—ε-malemidocaproyl-oxysuccinimide ester), GMBS (N-γ-maleimidobutyryl-oxysuccinimide ester), LC-SMCC (succinimidyl 4-(N-maleimidomethyl)cyclohexane-1-carboxy-(6-amidocaproate)), LC-SPDP (succinimidyl 6-(3(2-pyridyldithio)propionamido)hexanoate), MBS (m-maleimidobenzoyl-N-hydroxysuccinimide ester), PEG4-SPDP (PEGylated, long-chain SPDP crosslinker), SBAP (succinimidyl 3-(bromoacetamido)propionate), SIA (succinimidyl iodoacetate), SIAB (succinimidyl (4-iodoacetyl)aminobenzoate), SM(PEG)12 (PEGylated, long-chain SMCC crosslinker), SMPB (succinimidyl 4-(β-maleimidophenyl)butyrate), SMPH (Succinimidyl 6-((beta-maleimidopropionamido)hexanoate)), SMPT (4-succinimidyloxycarbonyl-alpha-methyl-α(2-pyridyldithio)toluene), SPDP (succinimidyl 3-(2-pyridyldithio)propionate), Sulfo-EMCS (N—ε-maleimidocaproyl-oxysulfosuccinimide ester), Sulfo-GMBS (N-γ-maleimidobutyryl-oxysulfosuccinimide ester), Sulfo-KMUS (N-κ-maleimidoundecanoyl-oxysulfosuccinimide ester), Sulfo-MBS (m-maleimidobenzoyl-N-hydroxysulfosuccinimide ester), Sulfo-SIAB (sulfosuccinimidyl (4-iodoacetyl)aminobenzoate), Sulfo-SMPB (sulfosuccinimidyl 4-(N-maleimidophenyl)butyrate), or a combination thereof. In still further embodiments, the thiol to thiol crosslinking agent is BM(PEG)3 (1,11-bismaleimido-triethyleneglycol), BMB (1,4-bismaleimidobutane), BMH (bismaleimidohexane), BMOE (bismaleimidoethane), DTME (dithiobismaleimidoethane), TMEA (tris(2-maleimidoethyl)amine), or a combination thereof.

As described herein, in various embodiments SNAs (e.g., CLSNAs) comprise various surface densities of oligonucleotides. In various embodiments, the ratio of oligonucleotide to tumor cell antigens is about 5:1 to about 1:5. In some embodiments, the ratio of oligonucleotide to tumor cell antigens is about 5:1, 4:1, 3:1, 2:1, 1.5:1, 1:1, 1:1.5, 1:2, 1:3, 1:4, or 1:5.

### Oligonucleotides

The disclosure provides cross-linked tumor lysate spherical nucleic acids (CLSNAs) comprising (a) a core comprising a plurality of cross-linked tumor cell antigens; and (b) a shell of oligonucleotides attached to the external surface of the core, the shell of oligonucleotides comprising one or more immunostimulatory oligonucleotides. In various embodiments, about or at least about 5%, 10%, 20%, 30%, 40%, 50%, 60%, 70% 80%, 90%, 95%, or 100% of oligonucleotides in the shell of oligonucleotides are immunostimulatory oligonucleotides. In various embodiments, the shell of oligonucleotides comprises an inhibitory oligonucleotide, a targeting oligonucleotide, a non-targeting oligonucleotide, or a combination thereof. Oligonucleotides contemplated for use according to the disclosure include those attached to the core through any means (e.g., covalent or non-covalent attachment). Oligonucleotides of the disclosure include, in various embodiments, DNA oligonucleotides, RNA oligonucleotides, modified forms thereof, or a combination thereof. In any aspects or embodiments described herein, an oligonucleotide is single-stranded, double-stranded, or partially double-stranded. In any aspects or embodiments of the disclosure, an oligonucleotide comprises a detectable marker (e.g., a fluorophore).

As described herein, modified forms of oligonucleotides are also contemplated by the disclosure which include those having at least one modified internucleotide linkage. In some embodiments, the oligonucleotide is all or in part a peptide nucleic acid. Other modified internucleoside linkages include at least one phosphorothioate linkage. Still other modified oligonucleotides include those comprising one or more universal bases. “Universal base” refers to molecules capable of substituting for binding to any one of A, C, G, T and U in nucleic acids by forming hydrogen bonds without significant structure destabilization. The oligonucleotide incorporated with the universal base analogues is able to function, e.g., as a probe in hybridization. Examples of universal bases include but are not limited to 5′-nitroindole-2′-deoxyriboside, 3-nitropyrrole, inosine and hypoxanthine.

The term “nucleotide” or its plural as used herein is interchangeable with modified forms as discussed herein and otherwise known in the art. The term “nucleobase” or its plural as used herein is interchangeable with modified forms as discussed herein and otherwise known in the art. Nucleotides or nucleobases comprise the naturally occurring nucleobases A, G, C, T, and U. Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C3-C6)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, oligonucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Examples of oligonucleotides include those containing modified backbones or non-natural internucleoside linkages. Oligonucleotides having modified backbones include those that retain a phosphorus atom in the backbone and those that do not have a phosphorus atom in the backbone. Modified oligonucleotides that do not have a phosphorus atom in their internucleoside backbone are considered to be within the meaning of “oligonucleotide”.

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are oligonucleotides having inverted polarity comprising a single 3′ to 3′ linkage at the 3′-most internucleotide linkage, i.e., a single inverted nucleoside residue which may be abasic (the nucleotide is missing or has a hydroxyl group in place thereof). Salts, mixed salts and free acid forms are also contemplated. Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In still further embodiments, oligonucleotide mimetics wherein both one or more sugar and/or one or more internucleotide linkage of the nucleotide units are replaced with “non-naturally occurring” groups. The bases of the oligonucleotide are maintained for hybridization. In some aspects, this embodiment contemplates a peptide nucleic acid (PNA).

In PNA compounds, the sugar-backbone of an oligonucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

In still further embodiments, oligonucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂— described in U.S. Pat. Nos. 5,489,677, and 5,602,240. Also contemplated are oligonucleotides with morpholino backbone structures described in U.S. Pat. No. 5,034,506.

In various forms, the linkage between two successive monomers in the oligonucleotide consists of 2 to 4, desirably 3, groups/atoms selected from —CH₂—, —O—, —S—, —NR^{H}—, >C═O, >C═NR^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHRH)—, where RH is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂— CH=(including R⁵ when used as a linkage to a succeeding monomer), —CH₂—CH₂—O—, —NR^{H}—CH₂—CH₂—, —CH₂—CH₂—NR^{H}—, —CH₂—NR^{H}—CH₂—, —O—CH₂—CH₂—NR^{H}—, —NR^{H}—CO—O—, —NR^{H}—CO—NR^{H}—, —NR^{H}—CS—NR^{H}—, —NR^{H}—C(═NR^{H})—NR^{H}—, —NR^{H}—CO—CH₂—NR^{H}—O—CO—O—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, —CH═N—O—, —CH₂—NR^{H}—O—, —CH₂—O—N═ (including R⁵ when used as a linkage to a succeeding monomer), —CH₂—O—NR^{H}—, —CO—NR^{H}—CH₂—, —CH₂—NR^{H}—O—, —CH₂—NR^{H}—CO—, —O—NR^{H}—CH₂—, —O—NR^{H}, —O—CH₂—S—, —S—CH₂—O—, —CH₂— CH₂—S—, —O—CH₂— CH₂—S—, —S—CH₂—CH=(including R⁵ when used as a linkage to a succeeding monomer), —S—CH₂— CH₂—, —S—CH₂— CH₂—O—, —S—CH₂— CH₂—S—, —CH₂—S—CH₂—, —CH₂—SO—CH₂—, —CH₂—SO₂—CH₂—, —O—SO—O—, —O—S(O)₂—O—, —O—S(O)₂— CH₂—, —O—S(O)₂—NR^{H}—, —NR^{H}—S(O)₂—CH₂—; —O—S(O)₂— CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂— S—, —S—P(O,S)—S—, —S—P(S)₂—S—, —O—PO(R″)—O—, —O—PO(OCH₃)—O—, —O—PO(O CH₂CH₃)—O—, —O—PO(O CH₂CH₂S—R)—O—, —O—PO(BH₃)—O—, —O—PO(NHR^{N})—O—, —O—P(O)₂—NR^{H} H—, —NR^{H}—P(O)₂—O—, —O—P(O,NR^{H})—O—, —CH₂—P(O)₂—O—, —O—P(O)₂— CH₂—, and —O—Si(R″)₂—O—; among which —CH₂— CO—NR^{H}—, —CH₂—NR^{H}—O—, —S—CH₂—O—, —O—P(O)₂—O—O—P(—O,S)—O—, —O—P(S)₂—O—, —NR^{H} P(O)₂—O—, —O—P(O,NR^{H})—O—, —O—PO(R″)—O—, —O—PO(CH₃)—O—, and —O—PO(NHR^{N})—O—, where RH is selected form hydrogen and C₁₋₄-alkyl, and R″ is selected from C₁₋₆-alkyl and phenyl, are contemplated. Further illustrative examples are given in Mesmaeker et. al., Current Opinion in Structural Biology 1995, 5, 343-355 and Susan M. Freier and Karl-Heinz Altmann, Nucleic Acids Research, 1997, vol 25, pp 4429-4443.

Still other modified forms of oligonucleotides are described in detail in U.S. patent application No. 20040219565, the disclosure of which is incorporated by reference herein in its entirety.

Modified oligonucleotides may also contain one or more substituted sugar moieties. In certain aspects, oligonucleotides comprise one of the following at the 2′ position: OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Other embodiments include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]2, where n and m are from 1 to about 10. Other oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, or an RNA cleaving group. In one aspect, a modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. Other modifications include 2′-dimethylaminooxyethoxy, i.e., a O(CH₂)₂ON(CH₃)₂ group, also known as 2′-DMAOE, and 2′-dimethylaminoethoxyethoxy (also known in the art as 2′—O-dimethyl-amino-ethoxy-ethyl or 2′-DMAEOE), i.e., 2′-O—CH₂—O—CH₂—N(CH₃)₂.

Still other modifications include 2′-methoxy (2′-O—CH₃), 2′-aminopropoxy (2′-OCH₂CH₂CH₂NH₂), 2′-allyl (2′-CH₂—CH═CH₂), 2′—O-allyl (2′-O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the oligonucleotide, for example, at the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In some aspects, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects is a methylene (—CH₂—)ₙ group bridging the 2′ oxygen atom and the 4′ carbon atom wherein n is 1 or 2. LNAs and preparation thereof are described in WO 98/39352 and WO 99/14226.

Modified nucleotides are described in EP 1 072 679 and WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing binding affinity and include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′—O-methoxyethyl sugar modifications. See, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

In various aspects, an oligonucleotide of the disclosure, or a modified form thereof, is generally about 5 nucleotides to about 1000 nucleotides in length. More specifically, an oligonucleotide of the disclosure is about 5 to about 1000 nucleotides in length, about 5 to about 900 nucleotides in length, about 5 to about 800 nucleotides in length, about 5 to about 700 nucleotides in length, about 5 to about 600 nucleotides in length, about 5 to about 500 nucleotides in length about 5 to about 450 nucleotides in length, about 5 to about 400 nucleotides in length, about 5 to about 350 nucleotides in length, about 5 to about 300 nucleotides in length, about 5 to about 250 nucleotides in length, about 5 to about 200 nucleotides in length, about 5 to about 150 nucleotides in length, about 5 to about 100, about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, about 10 to about 1000 nucleotides in length, about 10 to about 900 nucleotides in length, about 10 to about 800 nucleotides in length, about 10 to about 700 nucleotides in length, about 10 to about 600 nucleotides in length, about 10 to about 500 nucleotides in length about 10 to about 450 nucleotides in length, about 10 to about 400 nucleotides in length, about 10 to about 350 nucleotides in length, about 10 to about 300 nucleotides in length, about 10 to about 250 nucleotides in length, about 10 to about 200 nucleotides in length, about 10 to about 150 nucleotides in length, about 10 to about 100 nucleotides in length, about 10 to about 90 nucleotides in length, about 10 to about 80 nucleotides in length, about 10 to about 70 nucleotides in length, about 10 to about 60 nucleotides in length, about 10 to about 50 nucleotides in length about 10 to about 45 nucleotides in length, about 10 to about 40 nucleotides in length, about 10 to about 35 nucleotides in length, about 10 to about 30 nucleotides in length, about 10 to about 25 nucleotides in length, about 10 to about 20 nucleotides in length, about 10 to about 15 nucleotides in length, about 18 to about 28 nucleotides in length, about 15 to about 26 nucleotides in length, and all oligonucleotides intermediate in length of the sizes specifically disclosed to the extent that the oligonucleotide is able to achieve the desired result. In further embodiments, an oligonucleotide of the disclosure is about 5 to about 100 nucleotides in length, about 5 to about 90 nucleotides in length, about 5 to about 80 nucleotides in length, about 5 to about 70 nucleotides in length, about 5 to about 60 nucleotides in length, about 5 to about 50 nucleotides in length, about 5 to about 40 nucleotides in length, about 5 to about 30 nucleotides in length, about 5 to about 20 nucleotides in length, about 5 to about 10 nucleotides in length, and all oligonucleotides intermediate in length of the sizes specifically disclosed to the extent that the oligonucleotide is able to achieve the desired result. Accordingly, in various embodiments, an oligonucleotide of the disclosure is or is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 300, 400, 500, 600, 700, 800, 900, 1000, or more nucleotides in length. In further embodiments, an oligonucleotide of the disclosure is less than 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, or more nucleotides in length. In various embodiments, the shell of oligonucleotides attached to the exterior of the nanoparticle core of the SNA comprises a plurality of oligonucleotides that all have the same length/sequence, while in some embodiments, the plurality of oligonucleotides comprises one or more oligonucleotide that have a different length and/or sequence relative to at least one other oligonucleotide in the plurality. For example, and without limitation, in some embodiments the shell of oligonucleotides comprises a plurality of immunostimulatory oligonucleotides, wherein one immunostimulatory oligonucleotide has a sequence that is different than at least one other immunostimulatory oligonucleotide in the plurality.

In some embodiments, one or more oligonucleotides in the shell of oligonucleotides comprises or consists of a (GGX)ₙ nucleotide sequence, wherein n is 2-20 and X is a nucleobase (A, C, T, G, or U). In some embodiments, the (GGX)ₙ nucleotide sequence is on the 5′ end of the one or more oligonucleotides. In some embodiments, the (GGX)ₙ nucleotide sequence is on the 3′ end of the one or more oligonucleotides. In some embodiments, one or more oligonucleotides in the shell of oligonucleotides comprises or consists of a (GGT)ₙ nucleotide sequence, wherein n is 2-20. In some embodiments, the (GGT)ₙ nucleotide sequence is on the 5′ end of the one or more oligonucleotides. In some embodiments, the (GGT)ₙ nucleotide sequence is on the 3′ end of the one or more oligonucleotides.

In some embodiments, an oligonucleotide in the shell of oligonucleotides is a targeting oligonucleotide, such as an aptamer. Accordingly, all features and aspects of oligonucleotides described herein (e.g., length, type (DNA, RNA, modified forms thereof), optional presence of spacer) also apply to aptamers. Aptamers are oligonucleotide sequences that can be evolved to bind to various target analytes of interest. Aptamers may be single stranded, double stranded, or partially double stranded.

Spacers. In some aspects and embodiments, one or more oligonucleotides in the shell of oligonucleotides that is attached to the core of a CLSNA comprise a spacer. “Spacer” as used herein means a moiety that serves to increase distance between the core and the oligonucleotide, or to increase distance between individual oligonucleotides when attached to the core in multiple copies, or to improve the synthesis of the SNA. Thus, spacers are contemplated being located between an oligonucleotide and the core.

In some aspects, the spacer when present is an organic moiety. In some aspects, the spacer is a polymer, including but not limited to a water-soluble polymer, a nucleic acid, a polypeptide, an oligosaccharide, a carbohydrate, a lipid, an ethylglycol, or a combination thereof. In any of the aspects or embodiments of the disclosure, the spacer is an oligo(ethylene glycol)-based spacer. In various embodiments, an oligonucleotide comprises 1, 2, 3, 4, 5, or more spacer (e.g., Spacer-18 (hexaethyleneglycol)) moieties. In further embodiments, the spacer is an alkane-based spacer (e.g., C12). In some embodiments, the spacer is an oligonucleotide spacer (e.g., T5). An oligonucleotide spacer may have any sequence that does not interfere with the ability of the oligonucleotides to become bound to the nanoparticle core or to a target. In certain aspects, the bases of the oligonucleotide spacer are all adenylic acids, all thymidylic acids, all cytidylic acids, all guanylic acids, all uridylic acids, or all some other modified base.

In various embodiments, the length of the spacer is or is equivalent to at least about 2 nucleotides, at least about 3 nucleotides, at least about 4 nucleotides, at least about 5 nucleotides, 5-10 nucleotides, 10 nucleotides, 10-30 nucleotides, or even greater than 30 nucleotides.

SNA surface density. Generally, a surface density of oligonucleotides that is at least about 0.5 pmol/cm² will be adequate to provide a stable CLSNA. In further embodiments, a surface density of oligonucleotides that is at least about 1 pmol/cm², 1.5 pmol/cm², or 2 pmoles/cm² will be adequate to provide a stable CLSNA. In some aspects, the surface density of a CLSNA of the disclosure is at least 15 pmoles/cm². Methods are also provided wherein the oligonucleotide is attached to the core of the CLSNA at a surface density of about 0.5 pmol/cm² to about 1000 pmol/cm², or about 2 pmol/cm² to about 200 pmol/cm², or about 10 pmol/cm² to about 100 pmol/cm². In some embodiments, the surface density is about 1.7 pmol/cm². In some embodiments, the surface density is about 2 pmol/cm². In further embodiments, the surface density is at least about 0.5 pmol/cm², at least about 0.6 pmol/cm², at least about 0.7 pmol/cm², at least about 0.8 pmol/cm², at least about 0.9 pmol/cm², at least about 1 pmol/cm², at least about 1.5 pmol/cm², at least about 2 pmol/cm², at least 3 pmol/cm², at least 4 pmol/cm², at least 5 pmol/cm², at least 6 pmol/cm², at least 7 pmol/cm², at least 8 pmol/cm², at least 9 pmol/cm², at least 10 pmol/cm², at least about 15 pmol/cm², at least about 19 pmol/cm², at least about 20 pmol/cm², at least about 25 pmol/cm², at least about 30 pmol/cm², at least about 35 pmol/cm², at least about 40 pmol/cm², at least about 45 pmol/cm², at least about 50 pmol/cm², at least about 55 pmol/cm², at least about 60 pmol/cm², at least about 65 pmol/cm², at least about 70 pmol/cm², at least about 75 pmol/cm², at least about 80 pmol/cm², at least about 85 pmol/cm², at least about 90 pmol/cm², at least about 95 pmol/cm², at least about 100 pmol/cm², at least about 125 pmol/cm², at least about 150 pmol/cm², at least about 175 pmol/cm², at least about 200 pmol/cm², at least about 250 pmol/cm², at least about 300 pmol/cm², at least about 350 pmol/cm², at least about 400 pmol/cm², at least about 450 pmol/cm², at least about 500 pmol/cm², at least about 550 pmol/cm², at least about 600 pmol/cm², at least about 650 pmol/cm², at least about 700 pmol/cm², at least about 750 pmol/cm², at least about 800 pmol/cm², at least about 850 pmol/cm², at least about 900 pmol/cm², at least about 950 pmol/cm², at least about 1000 pmol/cm² or more. In further embodiments, the surface density is less than about 2 pmol/cm², less than about 3 pmol/cm², less than about 4 pmol/cm², less than about 5 pmol/cm², less than about 6 pmol/cm², less than about 7 pmol/cm², less than about 8 pmol/cm², less than about 9 pmol/cm², less than about 10 pmol/cm², less than about 15 pmol/cm², less than about 19 pmol/cm², less than about 20 pmol/cm², less than about 25 pmol/cm², less than about 30 pmol/cm², less than about 35 pmol/cm², less than about 40 pmol/cm², less than about 45 pmol/cm², less than about 50 pmol/cm², less than about 55 pmol/cm², less than about 60 pmol/cm², less than about 65 pmol/cm², less than about 70 pmol/cm², less than about 75 pmol/cm², less than about t 80 pmol/cm², less than about 85 pmol/cm², less than about 90 pmol/cm², less than about 95 pmol/cm², less than about 100 pmol/cm², less than about 125 pmol/cm², less than about 150 pmol/cm², less than about 175 pmol/cm², less than about 200 pmol/cm², less than about 250 pmol/cm², less than about 300 pmol/cm², less than about 350 pmol/cm², less than about 400 pmol/cm², less than about 450 pmol/cm², less than about 500 pmol/cm², less than about 550 pmol/cm², less than about 600 pmol/cm², less than about 650 pmol/cm², less than about 700 pmol/cm², less than about 750 pmol/cm², less than about 800 pmol/cm², less than about 850 pmol/cm², less than about 900 pmol/cm², less than about 950 pmol/cm², or less than about 1000 pmol/cm².

Alternatively, the density of oligonucleotide attached to the CLSNA is measured by the number of oligonucleotides attached to the CLSNA. With respect to the surface density of oligonucleotides attached to a CLSNA of the disclosure, it is contemplated that a CLSNA as described herein comprises or consists of about 1 to about 2,500, or about 1 to about 800 oligonucleotides on its surface. In various embodiments, a SNA comprises about 10 to about 800, or about 10 to about 750, or about 10 to about 700, or about 10 to about 600, or about 10 to about 500, or about 10 to about 300, or about 10 to about 200, or about 10 to about 190, or about 10 to about 180, or about 10 to about 170, or about 10 to about 160, or about 10 to about 150, or about 10 to about 140, or about 10 to about 130, or about 10 to about 120, or about 10 to about 110, or about 10 to about 100, or 10 to about 90, or about 10 to about 80, or about 10 to about 70, or about 10 to about 60, or about 10 to about 50, or about 10 to about 40, or about 10 to about 30, or about 10 to about 20, or about 75 to about 200, or about 75 to about 150, or about 100 to about 200, or about 150 to about 200 oligonucleotides in the shell of oligonucleotides attached to the core. In some embodiments, a CLSNA comprises about 200 to about 300 oligonucleotides in the shell of oligonucleotides attached to the core. In further embodiments, a CLSNA comprises at least about 5, 10, 20, 30, 40, 45, 50, 55, 60, 65, 70, 75, 80, 85, 90, 95, 100, 105, 110, 115, 120,125, 130, 135, 140, 145, 150, 155, 160,165, 170, 175, 180, 185,190, 195, 200, 220, 230, 240, 250, 260, 270, 280, 290, 300, 400, 500, 600, 700, or 800 oligonucleotides in the shell of oligonucleotides attached to the core. In further embodiments, a CLSNA consists of 5, 10, 20, 30, 40, 45, 50, 55, 60, 65, 70, 75, 80, 85, 90, 95, 100, 105, 110, 115, 120, 125, 130, 135, 140, 145, 150, 155, 160,165, 170, 175, 180, 185,190, 195, 200, 220, 230, 240, 250, 260, 270, 280, 290, 300, 400, 500, 600, 700, or 800 oligonucleotides in the shell of oligonucleotides attached to the core. In still further embodiments, the shell of oligonucleotides attached to the core of the CLSNA comprises 2, 3, 4, 5, 6, 7, 8, 9, 10, 11, 12, 13, 14, 15, 16, 17, 18, 19, 20, 50, 60, 70, 75, 80, 90, 100, 150, 160, 170, 175, 180, 190, 200, 220, 230, 240, 250, 260, 270, 280, 290, 300, 400, 500, 600, 700, 800 or more oligonucleotides. In some embodiments, the shell of oligonucleotides attached to the core of the CLSNA consists of 2, 3, 4, 5, 6, 7, 8, 9, 10, 11, 12, 13, 14, 15, 16, 17, 18, 19, 20, 30, 40, 50, 60, 70, 75, 80, 90, 100, 150, 160, 170, 175,180, 190, 200, 220, 230, 240, 250, 260, 270, 280, 290, 300, 400, 500, 600, 700, or 800 oligonucleotides. In some embodiments, the shell of oligonucleotides comprises about 200 to about 300 oligonucleotides.

### Uses of CLSNAS in Immune Regulation

Toll-like receptors (TLRs) are a class of proteins, expressed in sentinel cells, that play a key role in regulation of innate immune system. The mammalian immune system uses two general strategies to combat infectious diseases. Pathogen exposure rapidly triggers an innate immune response that is characterized by the production of immunostimulatory cytokines, chemokines and polyreactive IgM antibodies. The innate immune system is activated by exposure to Pathogen Associated Molecular Patterns (PAMPs) that are expressed by a diverse group of infectious microorganisms. The recognition of PAMPs is mediated by members of the Toll-like family of receptors. TLR receptors, such as TLR 8 and TLR 9 that respond to specific oligonucleotides are located inside special intracellular compartments, called endosomes. The mechanism of modulation of, for example and without limitation, TLR 8 and TLR 9 receptors, is based on DNA-protein interactions.

As described herein, immunostimulatory oligonucleotides that contain CpG motifs that are similar to those found in bacterial DNA stimulate a similar response of the TLR receptors. Thus, CpG oligonucleotides of the disclosure have the ability to function as TLR agonists. Other TLR agonists contemplated by the disclosure include, without limitation, single-stranded RNA and small molecules (e.g., R848 (Resiquimod)). Therefore, immunomodulatory (e.g., immunostimulatory) oligonucleotides have various potential therapeutic uses, including treatment of cancer.

Accordingly, in some embodiments, methods of utilizing CLSNAs as described herein for modulating toll-like receptors are disclosed. The methods up-regulate the Toll-like-receptor activity through the use of a TLR agonist. The method comprises contacting a cell having a toll-like receptor with a CLSNA of the disclosure, thereby modulating the activity and/or the expression of the toll-like receptor. The toll-like receptors modulated include one or more of toll-like receptor 1, toll-like receptor 2, toll-like receptor 3, toll-like receptor 4, toll-like receptor 5, toll-like receptor 6, toll-like receptor 7, toll-like receptor 8, toll-like receptor 9, toll-like receptor 10, toll-like receptor 11, toll-like receptor 12, and/or toll-like receptor 13.

### Methods of Inducing an Immune Response

The disclosure also includes methods for eliciting an immune response in a subject in need thereof, comprising administering to the subject an effective amount of a CLSNA (e.g., formulated as an antigenic composition) of the disclosure. In any of the aspects or embodiments of the disclosure, administering CLSNAs as described herein (e.g., formulated as a composition, pharmaceutical formulation, or antigenic composition) to a subject generates an antitumor response in the subject. In various embodiments, administering CLSNAs of the disclosure (e.g., formulated as a composition, pharmaceutical formulation, or antigenic composition) to a subject results in an increase in the amount of neutralizing antibodies against the antigen(s) that is produced in the subject relative to the amount of neutralizing antibodies against the antigen(s) that is produced in a subject who was not administered the CLSNAs. In further embodiments, the increase is a 2-fold increase, a 5-fold increase, a 10-fold increase, a 50-fold increase, a 100-fold increase, a 200-fold increase, a 500-fold increase, a 700-fold increase, or a 1000-fold increase.

In further embodiments, CLSNAs of the disclosure activate human peripheral blood mononuclear cells and generate an antibody response against one or more antigens as described herein. In some embodiments, the antibody response is a total antigen-specific antibody response. In further embodiments, administering CLSNAs of the disclosure (e.g., formulated as a composition, pharmaceutical formulation, or antigenic composition) to a subject results in an increase in the amount of total antigen-specific antibodies against the antigen(s) that is produced in the subject relative to the amount of total antigen-specific antibodies against the antigen(s) that is produced in a subject who was not administered the CLSNAs. In further embodiments, the increase is a 2-fold increase, a 5-fold increase, a 10-fold increase, a 50-fold increase, a 100-fold increase, a 200-fold increase, a 500-fold increase, a 700-fold increase, or a 1000-fold increase. A “total antigen-specific antibody response” is a measure of all of the antibodies (including neutralizing and non-neutralizing antibodies) that bind to a particular antigen.

The immune response raised by the methods of the present disclosure generally includes an antibody response, preferably a neutralizing antibody response, maturation and memory of T and B cells, antibody dependent cell-mediated cytotoxicity (ADCC), antibody cell-mediated phagocytosis (ADCP), complement dependent cytotoxicity (CDC), and T cell-mediated response such as CD4⁺, CD8⁺. The immune response generated by the SNA as disclosed herein generates an immune response and preferably treats a cancer as described herein. Methods for assessing antibody responses after administration of an antigenic composition (immunization or vaccination) are known in the art and/or described herein. In some embodiments, the immune response comprises a T cell-mediated response (e.g., peptide-specific response such as a proliferative response or a cytokine response). In any of the aspects or embodiments of the disclosure, the immune response comprises both a B cell and a T cell response. Antigenic compositions can be administered in a number of suitable ways, such as intramuscular injection, subcutaneous injection, intradermal administration and mucosal administration such as oral or intranasal. Additional modes of administration include but are not limited to intravenous, intraperitoneal, intranasal administration, intra-vaginal, intra-rectal, and oral administration. A combination of different routes of administration in the immunized subject, for example intramuscular and intranasal administration at the same time, is also contemplated by the disclosure.

Administration can involve a single dose or a multiple dose schedule. Multiple doses may be used in a primary immunization schedule and/or in a booster immunization schedule. In a multiple dose schedule the various doses may be given by the same or different routes, e.g., a parenteral prime and mucosal boost, a mucosal prime and parenteral boost, or a subcutaneous prime and a subcutaneous boost. Administration of more than one dose (typically two doses) is particularly useful in immunologically naive subjects or subjects of a hyporesponsive population (e.g., diabetics, or subjects with chronic kidney disease (e.g., dialysis patients)). In various embodiments, the second dose is administered about or at least about 2 weeks after the first dose. Multiple doses will typically be administered at least 1 week apart (e.g., about 2 weeks, about 3 weeks, about 4 weeks, about 6 weeks, about 8 weeks, about 10 weeks, about 12 weeks, or about 16 weeks). In some embodiments, multiple doses are administered from one, two, three, four or five months apart. Antigenic compositions of the present disclosure may be administered to patients at substantially the same time as (e.g., during the same medical consultation or visit to a healthcare professional) other vaccines.

### Uses of CLSNAS to Treat Cancer

In some aspects, a CLSNA of the disclosure is used to treat a cancer. Thus, in some aspects, the disclosure provides methods of treating a cancer comprising administering an effective amount of a CLSNA of the disclosure to a subject (e.g., a human subject) in need thereof, wherein the administering treats the cancer. In some aspects, the disclosure provides methods of treating a cancer comprising administering to a subject (e.g., a human subject) an effective amount of a CLSNA of the disclosure, thereby treating the cancer in the subject. In various embodiments, the cancer is bladder cancer, breast cancer, cervical cancer, colon cancer, rectal cancer, endometrial cancer, glioblastoma, kidney cancer, leukemia, liver cancer, lung cancer, melanoma, lymphoma, non-Hodgkin lymphoma, osteocarcinoma, ovarian cancer, pancreatic cancer, prostate cancer, thyroid cancer, and human papilloma virus-induced cancer, or a combination thereof.

### Additional Agents

In any of the aspects or embodiments of the disclosure, an additional agent is administered separately from a CLSNA of the disclosure. Thus, in various embodiments, a therapeutic agent is administered before, after, or concurrently with a CLSNA of the disclosure to treat a cancer.

In some aspects, the CLSNAs provided herein optionally further comprise an additional agent, or a plurality thereof. The additional agent is, in various embodiments, simply associated with an oligonucleotide in the shell of oligonucleotides attached to the core of the CLSNA, and/or the additional agent is associated with the core of the CLSNA. In some embodiments, the additional agent is associated with the end of an oligonucleotide in the shell of oligonucleotides that is not attached to the core (e.g., if the oligonucleotide is attached to the core through its 3′ end, then the additional agent is associated with the 5′ end of the oligonucleotide). Alternatively, in some embodiments, the additional agent is associated with the end of an oligonucleotide in the shell of oligonucleotides that is attached to the core (e.g., if the oligonucleotide is attached to the core through its 3′ end, then the additional agent is associated with the 3′ end of the oligonucleotide). In some embodiments, the additional agent is covalently associated with an oligonucleotide in the shell of oligonucleotides that is attached to the core of the CLSNA. In some embodiments, the additional agent is non-covalently associated with an oligonucleotide in the shell of oligonucleotides that is attached to the core of the CLSNA. However, it is understood that the disclosure provides CLSNAs wherein one or more additional agents are both covalently and non-covalently associated with oligonucleotides in the shell of oligonucleotides that is attached to the core of the SNA. It will also be understood that non-covalent associations include hybridization, protein binding, and/or hydrophobic interactions.

Additional agents contemplated by the disclosure include without limitation a protein (e.g., a therapeutic protein), a growth factor, a hormone, an interferon, an interleukin, an antibody or antibody fragment, a small molecule, a peptide, an antibiotic, an antifungal, an antiviral, a chemotherapeutic agent, or a combination thereof. In some embodiments, the additional agent is an anti-programmed cell death protein 1 (PD-1) antibody.

The term “small molecule,” as used herein, refers to a chemical compound or a drug, or any other low molecular weight organic compound, either natural or synthetic. By “low molecular weight” is meant compounds having a molecular weight of less than 1500 Daltons, typically between 100 and 700 Daltons.

### Uses of SNAs in Gene Regulation

In some aspects of the disclosure, an oligonucleotide associated with a CLSNA of the disclosure inhibits the expression of a gene. Thus, in some embodiments, a CLSNA performs both a vaccine function and a gene inhibitory function. In such aspects, the shell of oligonucleotides that is attached to the core of the CLSNA comprises one or more immunostimulatory oligonucleotides and one or more inhibitory oligonucleotides designed to inhibit target gene expression.

Methods for inhibiting gene product expression provided herein include those wherein expression of the target gene product is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% compared to gene product expression in the absence of a CLSNA. In other words, methods provided embrace those which results in essentially any degree of inhibition of expression of a target gene product.

The degree of inhibition is determined in vivo from a body fluid sample or from a biopsy sample or by imaging techniques well known in the art. Alternatively, the degree of inhibition is determined in a cell culture assay, generally as a predictable measure of a degree of inhibition that can be expected in vivo resulting from use of a specific type of CLSNA and a specific oligonucleotide.

In various aspects, the methods include use of an inhibitory oligonucleotide that is 100% complementary to the target polynucleotide, i.e., a perfect match, while in other aspects, the oligonucleotide is at least (meaning greater than or equal to) about 95% complementary to the polynucleotide over the length of the oligonucleotide, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20% complementary to the polynucleotide over the length of the oligonucleotide to the extent that the oligonucleotide is able to achieve the desired degree of inhibition of a target gene product.

The percent complementarity is determined over the length of the oligonucleotide. For example, given an antisense compound in which 18 of 20 nucleotides of the inhibitory oligonucleotide are complementary to a 20 nucleotide region in a target polynucleotide of 100 nucleotides total length, the oligonucleotide would be 90 percent complementary. In this example, the remaining noncomplementary nucleotides may be clustered or interspersed with complementary nucleobases and need not be contiguous to each other or to complementary nucleotides. Percent complementarity of an inhibitory oligonucleotide with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

The oligonucleotide utilized in such methods is either RNA or DNA. The RNA can be an inhibitory oligonucleotide, such as an inhibitory RNA (RNAi) that performs a regulatory function, and in various embodiments is selected from the group consisting of a small inhibitory RNA (siRNA), a single-stranded RNA (ssRNA), and a ribozyme. Alternatively, the RNA is microRNA that performs a regulatory function. The DNA is, in some embodiments, an antisense-DNA. In some embodiments, the RNA is a piwi-interacting RNA (piRNA).

The following examples are given merely to illustrate the present disclosure and not in any way to limit its scope.

### EXAMPLES

### Example 1

In this Example, successful synthesis and characterization of an SNA comprised of a covalently cross-linked tumor lysate core and an immunostimulatory oligonucleotide shell is demonstrated. This new lysate SNA exhibited the same advantageous properties, including high resistance to degradation in serum compared to the unprotected core or free tumor lysate, and increased uptake into bone marrow derived dendritic cells after only 30 minutes of incubation. Importantly, the synthesized SNA elevated immune activation of dendritic cells in vitro and in vivo.

Tumor cells (e.g., B16.F10) were collected from culture and suspended in Phosphate Buffer Saline (PBS). The cells were then lysed over 10 freeze-thaw cycles alternating between liquid nitrogen and sonication. The lysate was then spun at 10,000×g and the supernatant was collected. The lysate was then mixed with 100 fold excess of 4% paraformaldehyde by mass of protein. This mixture was shaken at 750 rpm for 30 minutes at room temperature, and then purified by 100 kDa cutoff spin filtration. Particle size and concentration (by molar) was characterized by nanoparticle tracking analysis. Particles were reacted with 1,000,000 fold molar excess of NHS-PEG4-Azide and shaken at room temperature for 1 hour. Excess NHS-PEG4-Azide was removed by spin filtration. Changes in particle size and concentration were characterized by nanoparticle tracking analysis. Particles were reacted with 1,000,000 fold molar excess of 5′ DBCO functionalized oligonucleotides and shaken at 750 rpm at room temperature overnight. Purification of synthesized SNAs was then accomplished using at least 10 cycles of spin filtration with PBS washes.

### Synthesis of Cross Linked Tumor Lysate Spherical Nucleic Acids (CLSNA)

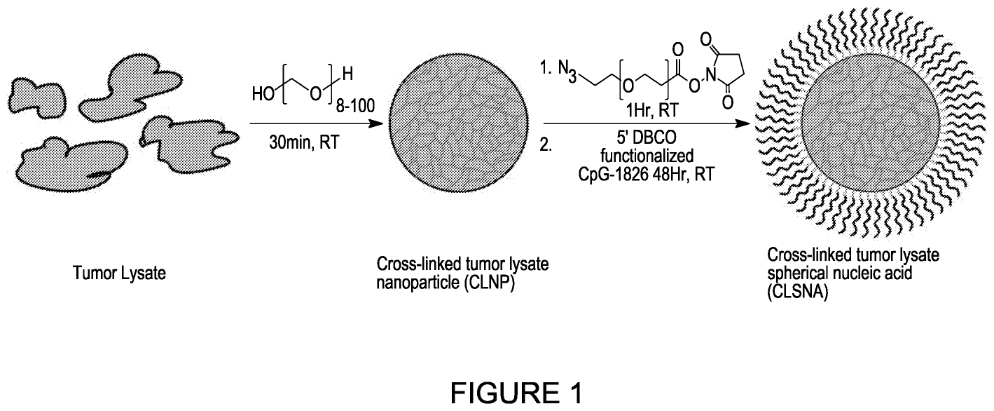

The synthesis of cross-linked tumor lysate spherical nucleic acids (CLSNA) begins with the lysis of tumor cells. These tumor cells are cultured in media and collected at high concentrations before being repeatedly cycled between liquid nitrogen and a sonicator; this process ruptures the tumor cells and leaves lysate. This lysate is then spun at high speeds to remove cellular debris before the lysate is collected from the supernatant. The tumor lysate is then reacted with 4% paraformaldehyde (100:1 molar excess), a long polymeric form of formaldehyde, that can cross-link to amino acids in the lysate that contain a primary amine. This mixture is shaken at 600-750 rpm for 30 minutes and produces the cross-linked tumor lysate nanoparticle (termed CLNP). These CLNPs are spun through a spin filter to remove any residual unreacted crosslinker. CLNPs are functionalized with an NHS-PEG4-Azide. The NHS ester can react with primary amines to form amide bonds and results in the CLNP being covered in surface azide groups. These azide groups can then react with DBCO-functionalized DNA. Immunostimulatory oligonucleotides (CpG-1826) containing 5′ terminal DBCO groups were reacted with the azide-functionalized CLNPs and shaken at room temperature for 48 hours to produce CLSNAs (FIG. 1).

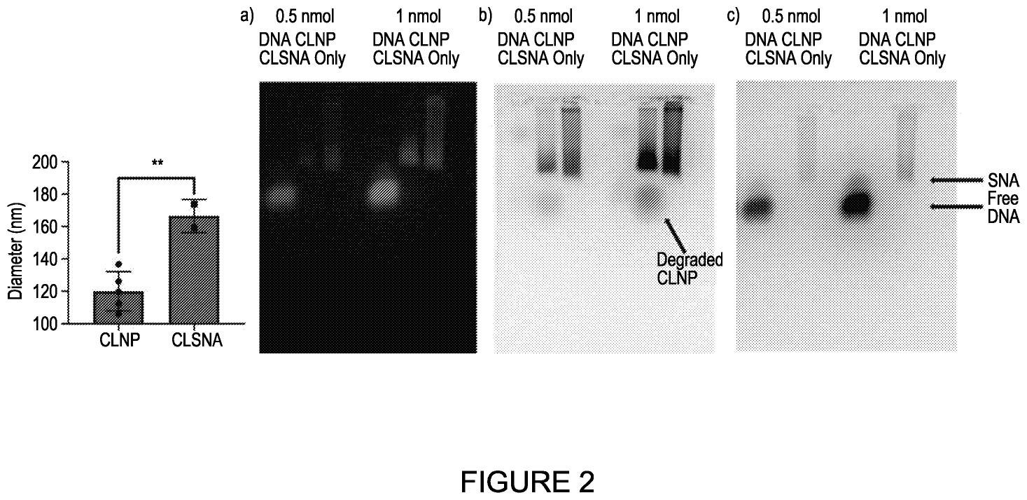

The left panel of FIG. 2 shows a comparison between cross-linked tumor lysate nanoparticles (CLNP) and cross-linked spherical nucleic acid (CLSNA) average particle diameter, collected by nanoparticle tracking analysis. CLSNAs display a size shift after the NHS-PEG4-azide and subsequent oligonucleotide functionalization. CLSNAs are approximately 170 nm in size. **p<0.01; n=2-5 per group. Statistical analysis was performed using two-tailed T-test. The right panel of FIG. 2 shows agarose gel electrophoresis of free DNA, CLNP, and CLSNA. Samples were loaded into the gels by dye at both 0.5 nmol and 1 nmol per lane. The core for both the CLNP and CLSNA was functionalized with an NHS-PEG4-Cy5 fluorophore, which causes the protein core to appear blue in FIG. 2A. The DNA shell on the CLSNA contained a Cy3 fluorophore, which appears red in FIG. 2A. FIG. 2A is a composite image of FIG. 2B and FIG. 2C. FIG. 2B was imaged in the Cy5 channel and demonstrates that the cross-linked protein core is present in both the CLNP and CLSNA but not in the DNA only lane. There is also a lighter band in the CLNP lane which appears to be degraded CLNP; this suggests that the CLNP is less resistant to degradation compared to CLSNA. FIG. 2C was imaged in the Cy3 channel and demonstrates that the oligonucleotides are present in both the DNA only lane as well as the CLSNA lane. CLSNA does not have a band at the same place as the free DNA, indicating that the mobility shift demonstrated by the Cy3 fluorophore corresponds to functionalization to the protein core.

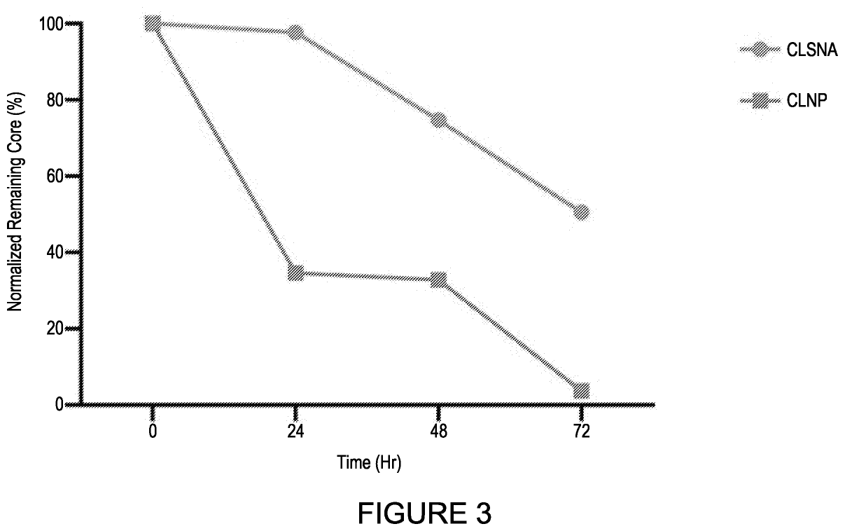

Fluorescently-labeled CLNP and CLSNA were incubated with 10% fetal bovine serum (FBS) and shaken at 37° C. Samples were collected at various time points and characterized by agarose gel electrophoresis. Band intensity was determined by ImageJ densitometry. Band intensity at the different time points was normalized to a 0 hr timepoint control. The rate of degradation was much more gradual for the CLSNA, with over 50% of the protein core remaining after 72 hrs in FBS compared to the CLNP which only had only approximately 3% of the protein core remaining at that same time point. See FIG. 3.

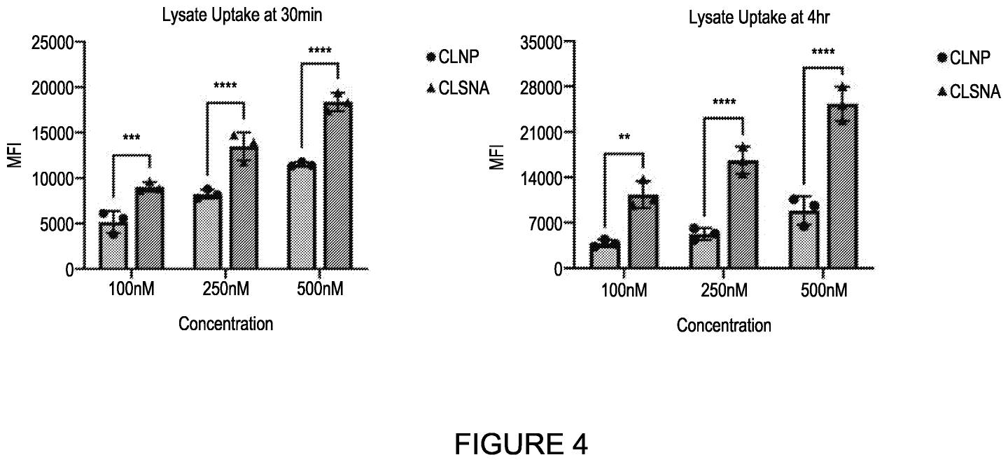

A key property of SNAs is enhanced and rapid uptake into cells through scavenger receptor A mediated endocytosis. In vitro experiments using bone marrow derived dendritic cells (BMDCs), a type of murine mortal immune cell essential to initiating a downstream immune response, was implemented to assess in vitro uptake. Both the CLNP and CLSNA were synthesized with Cy5 labeled protein cores for tracking via flow cytometry. Greater Median Fluorescence Intensity (MFI) of the Cy5 signal, as shown, indicates greater particle internalization into the BMDCs. The left panel of FIG. 4 shows uptake of CLNP or CLSNA after 30 min of incubation with BMDCs. CLSNA treatment resulted in significantly higher MFI at all tested protein concentrations (100 nM (P value <0.001), 250 nM (P value <0.0001), and 500 nM (P value <0.0001)). Statistical analysis completed by way of 2-way ANOVA followed by Sidak's multiple comparisons test. The right panel of FIG. 4 shows uptake of CLNP or CLSNA after 4 h of incubation with BMDCs. CLSNA treatment resulted in significantly higher MFI at all tested protein concentrations (100 nM (P value <0.01), 250 nM (P value <0.0001), and 500 nM (P value <0.0001)). Statistical analysis was completed by way of 2-way ANOVA followed by Sidak's multiple comparisons test.

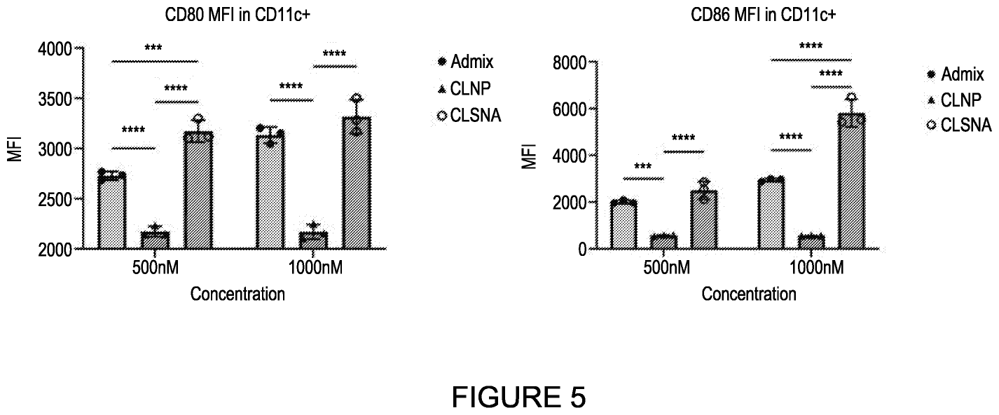

The ability for synthesized CLSNAs to stimulate the immune system in vitro was next assessed. CLSNA was compared against CLNP as well as admix (a simple mixture of the same DNA and lysate components in saline). Samples were incubated with BMDCs for 24 h. Immune activation was measured by expression of two different co-stimulatory markers, CD80 (left) and CD86 (right), via flow cytometry. Increase in MFI indicates greater expression of CD80 and CD86 and thus enhanced immune activation. The left panel of FIG. 5 shows expression level of costimulatory marker CD80 by BMDCs after in vitro incubation. CLSNA generated significantly greater CD80 expression at 500 nM (P value <0.001) protein concentration compared to admix. CLSNA also resulted in significantly greater CD80 expression at 500 nM (P value <0.0001) and 1000 nM (P value <0.0001) protein concentrations compared to CLNP. Statistical analysis was completed by way of 2-way ANOVA followed by Tukey's multiple comparisons test. The right panel of FIG. 5 shows expression of costimulatory marker CD86 by BMDCs after in vitro incubation. CLSNA produced significantly greater CD86 expression at 1000 nM (P value <0.0001) protein concentrations compared to admix. CLSNA also resulted in significantly greater CD86 expression at 500 nM (P value <0.0001) and 1000 nM (P value <0.0001) protein concentrations compared to CLNP. Statistical analysis was completed by way of 2-way ANOVA followed by Tukey's multiple comparisons test.

### REFERENCES

1. Berti, C.; Boarino, A.; Graciotti, M.; Bader, L. P. E.; Kandalaft, L. E.; Klok, H.-A. ReductionSensitive Protein Nanogels Enhance Uptake of Model and Tumor Lysate Antigens In Vitro by Mouse- and Human-Derived Dendritic Cells. ACS Appl. Bio Mater. 2021, 4 (12), 8291-8300. https://doi.org/10.1021/acsabm.1c00828.
2. Tang, L.; Zheng, Y.; Melo, M. B.; Mabardi, L.; Castano, A. P.; Xie, Y.-Q.; Li, N.; Kudchodkar, S. B.; Wong, H. C.; Jeng, E. K.; Maus, M. V.; Irvine, D. J. Enhancing T Cell Therapy through TCR-Signaling-Responsive Nanoparticle Drug Delivery. Nature Biotechnology 2018, 36 (8), 707-716. https://doi.org/10.1038/nbt.4181.
3. Tsoras, A. N.; Champion, J. A. Cross-Linked Peptide Nanoclusters for Delivery of Oncofetal Antigen as a Cancer Vaccine. Bioconjugate Chem. 2018, 29 (3), 776-785. https://doi.org/10.1021/acs.bioconjchem.8b00079.

What is claimed is:

1. A cross-linked tumor lysate spherical nucleic acid (CLSNA) comprising:
(a) a core comprising a plurality of cross-linked tumor cell antigens; and
(b) a shell of oligonucleotides attached to the external surface of the core, the shell of oligonucleotides comprising one or more immunostimulatory oligonucleotides.

2. The CLSNA of claim 1, wherein the plurality of cross-linked tumor cell antigens comprises a tumor cell lysate, purified protein tumor antigens, synthesized tumor antigens, or a combination thereof.

3. The CLSNA of claim 1 or claim 2, wherein the core comprises about 0.25 femtograms (fg)-2 fg of cross-linked tumor cell antigens.

4. The CLSNA of any one of claims 1-3, wherein at least one of the one or more immunostimulatory oligonucleotide comprises a CpG nucleotide sequence.

5. The CLSNA of any one of claims 1-4, wherein at least one of the one or more immunostimulatory oligonucleotides is a toll-like receptor (TLR) agonist.

6. The CLSNA of any one of claims 1-5, wherein each of the one or more immunostimulatory oligonucleotides is a toll-like receptor (TLR) agonist.

7. The CLSNA of claim 5 or claim 6, wherein the TLR agonist is a toll-like receptor 1 (TLR1) agonist, a toll-like receptor 2 (TLR2) agonist, a toll-like receptor 3 (TLR3) agonist, a toll-like receptor 4 (TLR4) agonist, a toll-like receptor 5 (TLR5) agonist, a toll-like receptor 6 (TLR6) agonist, a toll-like receptor 7 (TLR7) agonist, a toll-like receptor 8 (TLR8) agonist, a toll-like receptor 9 (TLR9) agonist, a toll-like receptor 10 (TLR10) agonist, a toll-like receptor 11 (TLR11) agonist, a toll-like receptor 12 (TLR12) agonist, a toll-like receptor 13 (TLR13) agonist, or a combination thereof.

8. The CLSNA of any one of claims 5-7, wherein the TLR agonist is a toll-like receptor 3 (TLR3) agonist, a toll-like receptor 7 (TLR7) agonist, a toll-like receptor 8 (TLR8) agonist, a toll-like receptor 9 (TLR9) agonist, or a combination thereof.

9. The CLSNA of any one of claims 1-8, wherein one or more oligonucleotides in the shell of oligonucleotides comprises or consists of the sequence of 5′-TCCATGACGTTCCTGACGTT-3′ (SEQ ID NO: 2).

10. The CLSNA of any one of claims 1-9, wherein one or more oligonucleotides in the shell of oligonucleotides comprises or consists of the sequence of 5′-TCGTCGTTTTGTCGTTTTGTCGTT-3′ (SEQ ID NO: 3).

11. The CLSNA of any one of claims 1-10, wherein one or more oligonucleotides in the shell of oligonucleotides comprises or consists of the sequence of 5′-TCCATGACGTTCCTGACGTT (Spacer-18 (hexaethyleneglycol))₂dibenzocyclooctyl (DBCO)-3′ (SEQ ID NO: 4).

12. The CLSNA of any one of claims 1-11, wherein one or more oligonucleotides in the shell of oligonucleotides comprises or consists of the sequence of 5′-TCGTCGTTTTGTCGTTTTGTCGTT (Spacer-18 (hexaethyleneglycol))₂dibenzocyclooctyl (DBCO)-3′ (SEQ ID NO: 5).

13. The CLSNA of any one of claims 1-12, wherein one or more oligonucleotides in the shell of oligonucleotides is modified on its 5′ end and/or 3′ end with dibenzocyclooctyl (DBCO).

14. The CLSNA of any one of claims 1-13, wherein one or more oligonucleotides in the shell of oligonucleotides is modified on its 5′ end and/or 3′ end with a thiol.

15. The CLSNA of any one of claims 1-13, wherein one or more oligonucleotides in the shell of oligonucleotides is modified on its 5′ end and/or 3′ end with a maleimide.

16. The CLSNA of any one of claims 1-13, wherein one or more oligonucleotides in the shell of oligonucleotides is modified on its 5′ end and/or 3′ end with an azide.

17. The CLSNA of any one of claims 1-16, wherein diameter of the CLSNA is about 20 nanometers (nm) to about 300 nm.

18. The CLSNA of any one of claims 1-17, wherein diameter of the CLSNA is less than or equal to about 300 nanometers.

19. The CLSNA of any one of claims 1-17, wherein diameter of the nanoparticle is less than or equal to about 170 nanometers.

20. The CLSNA of any one of claims 1-19, wherein the CLSNA comprises about 10 to about 750 oligonucleotides.

21. The CLSNA of claim 20, wherein the CLSNA comprises about 200 to about 300 oligonucleotides.

22. The CLSNA of any one of claims 1-21, wherein the plurality of cross-linked tumor cell antigens is derived from a tumor lysate exposed to a crosslinking agent.

23. The CLSNA of claim 22, wherein the crosslinking agent is an amine to amine, amine to thiol, thiol to thiol crosslinking agent, or a combination thereof.

24. The CLSNA of claim 23, wherein the amine to amine crosslinking agent is DSS (disuccinimidyl suberate), BS3 (bis(sulfosuccinimidyl)suberate), DSBU (Disuccinimidyl Dibutyric Urea), DFDNB (1,5-difluoro-2,4,dinitrobenzene), DMP (dimethyl pimelimidate), DMS (dimethyl suberimidate), DSG (disuccinimidyl glutarate), DSP (dithiobis(succinimidyl propionate)), DSSO (disuccinimidyl sulfoxide), DST (disuccinimidyl tartrate), DTBP (dimethyl 3,3′-dithiobispropionimidate), DTSSP (3,3′-dithiobis(sulfosuccinimidyl propionate)), EGS (ethylene glycol bis(succinimidyl succinate)), Sulfo-EGS (ethylene glycol bis(sulfosuccinimidyl succinate)), TSAT (tris-(succinimidyl)aminotriacetate), or a combination thereof.

25. The CLSNA of claim 23 or claim 24, wherein the amine to thiol crosslinking agent is Sulfo-SMCC (sulfosuccinimidyl 4-(N-maleimidomethyl)cyclohexane-1-carboxylate), SM(PEG)2 (PEGylated SMCC crosslinker), BMPS (N-β-maleimidopropyl-oxysuccinimide ester), AMAS (N-α-maleimidoacet-oxysuccinimide ester), EMCS (N—ε-malemidocaproyl-oxysuccinimide ester), GMBS (N-γ-maleimidobutyryl-oxysuccinimide ester), LC-SMCC (succinimidyl 4-(N-maleimidomethyl)cyclohexane-1-carboxy-(6-amidocaproate)), LC-SPDP (succinimidyl 6-(3(2-pyridyldithio)propionamido)hexanoate), MBS (m-maleimidobenzoyl-N-hydroxysuccinimide ester), PEG4-SPDP (PEGylated, long-chain SPDP crosslinker), SBAP (succinimidyl 3-(bromoacetamido)propionate), SIA (succinimidyl iodoacetate), SIAB (succinimidyl (4-iodoacetyl)aminobenzoate), SM(PEG)12 (PEGylated, long-chain SMCC crosslinker), SMPB (succinimidyl 4-(β-maleimidophenyl)butyrate), SMPH (Succinimidyl 6-((beta-maleimidopropionamido)hexanoate)), SMPT (4-succinimidyloxycarbonyl-alpha-methyl-α(2-pyridyldithio)toluene), SPDP (succinimidyl 3-(2-pyridyldithio)propionate), Sulfo-EMCS (N—ε-maleimidocaproyl-oxysulfosuccinimide ester), Sulfo-GMBS (N-γ-maleimidobutyryl-oxysulfosuccinimide ester), Sulfo-KMUS (N-κ-maleimidoundecanoyl-oxysulfosuccinimide ester), Sulfo-MBS (m-maleimidobenzoyl-N-hydroxysulfosuccinimide ester), Sulfo-SIAB (sulfosuccinimidyl (4-iodoacetyl)aminobenzoate), Sulfo-SMPB (sulfosuccinimidyl 4-(N-maleimidophenyl)butyrate), or a combination thereof.

26. The CLSNA of any one of claims 23-25, wherein the thiol to thiol crosslinking agent is BM(PEG)3 (1,11-bismaleimido-triethyleneglycol), BMB (1,4-bismaleimidobutane), BMH (bismaleimidohexane), BMOE (bismaleimidoethane), DTME (dithiobismaleimidoethane), TMEA (tris(2-maleimidoethyl)amine), or a combination thereof.

27. The CLSNA of any one of claims 1-26, wherein the plurality of cross-linked tumor cell antigens is derived from a breast cancer cell, peritoneum cancer cell, cervical cancer cell, colon cancer cell, rectal cancer cell, esophageal cancer cell, eye cancer cell, liver cancer cell, pancreatic cancer cell, larynx cancer cell, lung cancer cell, skin cancer cell, ovarian cancer cell, prostate cancer cell, stomach cancer cell, testicular cancer cell, thyroid cancer cell, brain cancer cell, or a combination thereof.

28. The CLSNA of any one of claims 1-27, wherein the shell of oligonucleotides comprises DNA oligonucleotides, RNA oligonucleotides, or a combination thereof.

29. The CLSNA of any one of claims 1-28, wherein the shell of oligonucleotides comprises DNA oligonucleotides and RNA oligonucleotides.

30. The CLSNA of any one of claims 1-29, wherein the shell of oligonucleotides comprises single-stranded DNA, double-stranded DNA, single-stranded RNA, double-stranded RNA, or a combination thereof.

31. The CLSNA of any one of claims 1-30, wherein the shell of oligonucleotides comprises a targeting oligonucleotide, an inhibitory oligonucleotide, a non-targeting oligonucleotide, or a combination thereof.

32. The CLSNA of claim 31, wherein the inhibitory oligonucleotide is an antisense oligonucleotide, small interfering RNA (siRNA), an aptamer, a short hairpin RNA (shRNA), a DNAzyme, or an aptazyme.

33. The CLSNA of any one of claims 1-32, wherein one or more oligonucleotides in the shell of oligonucleotides is a modified oligonucleotide.

34. The CLSNA of any one of claims 1-33, wherein each tumor cell antigen in the plurality of cross-linked tumor cell antigens is the same, or wherein at least two tumor cell antigens in the plurality of cross-linked tumor cell antigens are different.

35. A pharmaceutical formulation comprising the CLSNA of any one of claims 1-34 and a pharmaceutically acceptable carrier or diluent.

36. A method of making a cross-linked tumor lysate spherical nucleic acid (CLSNA) comprising:
contacting one or more tumor antigens with a crosslinking agent to produce a core comprising a plurality of cross-linked tumor cell antigens; then
contacting the core with one or more oligonucleotides to make the CLSNA, wherein the core and the one or more oligonucleotides comprise complementary reactive moieties that together form a covalent bond.

37. The method of claim 36, wherein the crosslinking agent is formaldehyde or paraformaldehyde.

38. The method of claim 36 or claim 37, wherein the reactive moiety on the core comprises an azide, an alkyne, a maleimide, a thiol, an alcohol, an amine, a carboxylic acid, an olefin, an isothiocyanate, a N-hydroxysuccinimide, a phosphine, a nitrone, a norbornene, an oxanorbornene, a transcycloctene, an s-tetrazene, an isocyanide, a tetrazole, a nitrile oxide, a quadricyclane, or a carbodiimide.

39. The method of any one of claims 36-38, wherein the reactive moiety is on a terminus of each of the one or more oligonucleotides.

40. The method of any one of claims 36-39, wherein the reactive moiety on the one or more oligonucleotides comprises an alkyne, an azide, a maleimide, a thiol, an alcohol, an amine, a carboxylic acid, an olefin, an isothiocyanate, a N-hydroxysuccinimide, a phosphine, a nitrone, a norbornene, an oxanorbornene, a transcycloctene, an s-tetrazene, an isocyanide, a tetrazole, a nitrile oxide, a quadricyclane, or a carbodiimide.

41. The method of any one of claims 38-40, wherein the alkyne comprises dibenzocyclooctyl (DBCO) alkyne or a terminal alkyne.

42. The method of any one of claims 38-41, wherein the core comprises an azide reactive moiety and each of the one or more oligonucleotides comprises an alkyne reactive moiety, or vice versa.

43. The method of claim 42, wherein the alkyne reactive moiety comprises a DBCO alkyne.

44. The method of any one of claims 36-43, wherein the one or more oligonucleotides comprises at least one Toll-Like Receptor (TLR) agonist.

45. The method of claim 44, wherein the TLR agonist is a toll-like receptor 1 (TLR1) agonist, a toll-like receptor 2 (TLR2) agonist, a toll-like receptor 3 (TLR3) agonist, a toll-like receptor 4 (TLR4) agonist, a toll-like receptor 5 (TLR5) agonist, a toll-like receptor 6 (TLR6) agonist, a toll-like receptor 7 (TLR7) agonist, a toll-like receptor 8 (TLR8) agonist, a toll-like receptor 9 (TLR9) agonist, a toll-like receptor 10 (TLR10) agonist, a toll-like receptor 11 (TLR11) agonist, a toll-like receptor 12 (TLR12) agonist, a toll-like receptor 13 (TLR13) agonist, or a combination thereof.

46. The method of claim 44 or claim 45, wherein the TLR agonist is a toll-like receptor 3 (TLR3) agonist, a toll-like receptor 7 (TLR7) agonist, a toll-like receptor 8 (TLR8) agonist, a toll-like receptor 9 (TLR9) agonist, or a combination thereof.

47. The method of any one of claims 36-46, wherein at least one of the one or more oligonucleotides comprises RNA or DNA.

48. The method of claim 47, wherein at least one of the one or more oligonucleotides is DNA.

49. The method of any one of claims 36-48, wherein at least one of the one or more oligonucleotides is a modified oligonucleotide.

50. The method of any one of claims 36-49, wherein the ratio of oligonucleotide to cross-linked tumor cell antigens is about 1:1 to about 1:2.

51. A vaccine comprising the CLSNA of any one of claims 1-34 or the pharmaceutical formulation of claim 35.

52. The vaccine of claim 51, comprising an adjuvant.

53. An antigenic composition comprising the CLSNA of any one of claims 1-34 in a pharmaceutically acceptable carrier, diluent, stabilizer, preservative, or adjuvant, the pharmaceutical formulation of claim 35, or the vaccine of claim 51 or 52, wherein the antigenic composition is capable of generating an immune response including antibody generation, an antitumor response, and/or a protective immune response in a mammalian subject.

54. The antigenic composition of claim 53, wherein the antibody response is a neutralizing antibody response or a protective antibody response.

55. A method of producing an immune response in a subject having cancer or at risk of developing cancer, comprising administering to the subject an effective amount of the CLSNA of any one of claims 1-34, the pharmaceutical formulation of claim 35, the vaccine of claim 51 or claim 52, or the antigenic composition of claim 53 or claim 54, thereby producing the immune response in the subject.

56. The method of claim 55, wherein the cancer is breast cancer, peritoneum cancer, cervical cancer, colon cancer, rectal cancer, esophageal cancer, eye cancer, liver cancer, pancreatic cancer, larynx cancer, lung cancer, skin cancer, ovarian cancer, prostate cancer, stomach cancer, testicular cancer, thyroid cancer, brain cancer, or a combination thereof.

57. A method of treating a cancer in a subject in need thereof, comprising administering to the subject an effective amount of the CLSNA of any one of claims 1-34, the pharmaceutical formulation of claim 35, the vaccine of claim 51 or claim 52, or the antigenic composition of claim 53 or claim 54, thereby treating the cancer in the subject.

58. The method of claim 57, wherein the cancer is breast cancer, peritoneum cancer, cervical cancer, colon cancer, rectal cancer, esophageal cancer, eye cancer, liver cancer, pancreatic cancer, larynx cancer, lung cancer, skin cancer, ovarian cancer, prostate cancer, stomach cancer, testicular cancer, thyroid cancer, brain cancer, or a combination thereof.

59. The method of claim 57 or claim 58, wherein the administering is subcutaneous.

60. The method of claim 57 or claim 58, wherein the administering is intravenous, intraperitoneal, intranasal, or intramuscular.


SEQUENCE LISTING (5 sequences)

SEQ ID NO: 1 - DNA, 20 bp, synthetic construct
  source 1..20: mol_type = other DNA
tttttttttttttttttttt

SEQ ID NO: 2 - DNA, 20 bp, synthetic construct
  source 1..20: mol_type = other DNA
tccatgacgttcctgacgtt

SEQ ID NO: 3 - DNA, 24 bp, synthetic construct
  source 1..24: mol_type = other DNA
tcgtcgttttgtcgttttgtcgtt

SEQ ID NO: 4 - DNA, 20 bp, synthetic construct
  source 1..20: mol_type = other DNA
  modified_base 20: mod_base = OTHER; note = Spacer-18 (hexaethyleneglycol)2dibenzocyclooctyl (DBCO))
tccatgacgttcctgacgtt

SEQ ID NO: 5 - DNA, 24 bp, synthetic construct
  source 1..24: mol_type = other DNA
  modified_base 20: mod_base = OTHER; note = Spacer-18 (hexaethyleneglycol)2dibenzocyclooctyl (DBCO))
tcgtcgttttgtcgttttgtcgtt